SEQUENCE LISTING (60 sequences)

SEQ ID NO: 1 - DNA, 69 bp, Artificial sequence
  Description of sequence ADTA1 antigen Hu. CD19
ccttggcacccgagaattccactgggcaattactcgbaaaaaaaaaaaaaaaaaaaaaaaaaaaaaaaa

SEQ ID NO: 2 - DNA, 69 bp, Artificial sequence
  Description of sequence ADTA2 antigen Hu.CD3
ccttggcacccgagaattccactcattgtaactcctbaaaaaaaaaaaaaaaaaaaaaaaaaaaaaaaa

SEQ ID NO: 3 - DNA, 69 bp, Artificial sequence
  Description of sequence ADTA3 antigen Hu. CD16
ccttggcacccgagaattccaaagttcactctttgcbaaaaaaaaaaaaaaaaaaaaaaaaaaaaaaaa

SEQ ID NO: 4 - DNA, 69 bp, Artificial sequence
  Description of sequence ADTA4 antigen Hu.CD4
ccttggcacccgagaattccatgttcccgctcaactbaaaaaaaaaaaaaaaaaaaaaaaaaaaaaaaa

SEQ ID NO: 5 - DNA, 69 bp, Artificial sequence
  Description of sequence ADTA5 antigen Hu. CD11c
ccttggcacccgagaattccatacgcctataacttgbaaaaaaaaaaaaaaaaaaaaaaaaaaaaaaaa

SEQ ID NO: 6 - DNA, 69 bp, Artificial sequence
  Description of sequence ADTA6 antigen Hu. CD56
ccttggcacccgagaattccatcctttcctgataggbaaaaaaaaaaaaaaaaaaaaaaaaaaaaaaaa

SEQ ID NO: 7 - DNA, 69 bp, Artificial sequence
  Description of sequence ADTA7 antigen Hu. CD14
ccttggcacccgagaattccatctcagacctccgtabaaaaaaaaaaaaaaaaaaaaaaaaaaaaaaaa

SEQ ID NO: 8 - DNA, 69 bp, Artificial sequence
  Description of sequence ADTA8 antigen Hu. CD8
ccttggcacccgagaattccagcgcaacttgatgatbaaaaaaaaaaaaaaaaaaaaaaaaaaaaaaaa

SEQ ID NO: 9 - DNA, 69 bp, Artificial sequence
  Description of sequence ADTA9 antigen Hu. CD45
ccttggcacccgagaattccatcccttgcgatttacbaaaaaaaaaaaaaaaaaaaaaaaaaaaaaaaa

SEQ ID NO: 10 - DNA, 69 bp, Artificial sequence
  Description of sequence ADTA10 antigen Hu. ZAP70
ccttggcacccgagaattccacttgcctgcaatcacbaaaaaaaaaaaaaaaaaaaaaaaaaaaaaaaa

SEQ ID NO: 11 - DNA, 69 bp, Artificial sequence
  Description of sequence ADTA11 antigen Hu. Perforin
ccttggcacccgagaattccatcttcgtttgaacggbaaaaaaaaaaaaaaaaaaaaaaaaaaaaaaaa

SEQ ID NO: 12 - DNA, 69 bp, Artificial sequence
  Description of sequence ADTA12 antigen Hu. GrzmB
ccttggcacccgagaattccagcgtgttgtggtattbaaaaaaaaaaaaaaaaaaaaaaaaaaaaaaaa

SEQ ID NO: 13 - DNA, 90 bp, Artificial sequence
  Description of sequence ADTB1 antigen Hu. CD19
  misc_feature (35)..(44): n is a, c, g, or t
  misc_feature (60)..(68): n is a, c, g, or t
gtgactggagttcagacgtgtgctcttccgatctnnnnnnnnnnctgggcaattactcgnnnnnnnnngctttaaggccggtcctagcaa

SEQ ID NO: 14 - DNA, 90 bp, Artificial sequence
  Description of sequence ADTB2 antigen Hu.CD3
  misc_feature (35)..(44): n is a, c, g, or t
  misc_feature (60)..(68): n is a, c, g, or t
gtgactggagttcagacgtgtgctcttccgatctnnnnnnnnnnctcattgtaactcctnnnnnnnnngctttaaggccggtcctagcaa

SEQ ID NO: 15 - DNA, 90 bp, Artificial sequence
  Description of sequence ADTB3 antigen Hu. CD16
  misc_feature (35)..(44): n is a, c, g, or t
  misc_feature (60)..(68): n is a, c, g, or t
gtgactggagttcagacgtgtgctcttccgatctnnnnnnnnnnaagttcactctttgcnnnnnnnnngctttaaggccggtcctagcaa

SEQ ID NO: 16 - DNA, 90 bp, Artificial sequence
  Description of sequence ADTB4 antigen Hu.CD4
  misc_feature (35)..(44): n is a, c, g, or t
  misc_feature (60)..(68): n is a, c, g, or t
gtgactggagttcagacgtgtgctcttccgatctnnnnnnnnnntgttcccgctcaactnnnnnnnnngctttaaggccggtcctagcaa

SEQ ID NO: 17 - DNA, 90 bp, Artificial sequence
  Description of sequence ADTB5 antigen Hu. CD11c
  misc_feature (35)..(44): n is a, c, g, or t
  misc_feature (60)..(68): n is a, c, g, or t
gtgactggagttcagacgtgtgctcttccgatctnnnnnnnnnntacgcctataacttgnnnnnnnnngctttaaggccggtcctagcaa

SEQ ID NO: 18 - DNA, 90 bp, Artificial sequence
  Description of sequence ADTB6 antigen Hu. CD56
  misc_feature (35)..(44): n is a, c, g, or t
  misc_feature (60)..(68): n is a, c, g, or t
gtgactggagttcagacgtgtgctcttccgatctnnnnnnnnnntcctttcctgataggnnnnnnnnngctttaaggccggtcctagcaa

SEQ ID NO: 19 - DNA, 90 bp, Artificial sequence
  Description of sequence ADTB7 antigen Hu. CD14
  misc_feature (35)..(44): n is a, c, g, or t
  misc_feature (60)..(68): n is a, c, g, or t
gtgactggagttcagacgtgtgctcttccgatctnnnnnnnnnntctcagacctccgtannnnnnnnngctttaaggccggtcctagcaa

SEQ ID NO: 20 - DNA, 90 bp, Artificial sequence
  Description of sequence ADTB8 antigen Hu.CD8
  misc_feature (35)..(44): n is a, c, g, or t
  misc_feature (60)..(68): n is a, c, g, or t
gtgactggagttcagacgtgtgctcttccgatctnnnnnnnnnngcgcaacttgatgatnnnnnnnnngctttaaggccggtcctagcaa

SEQ ID NO: 21 - DNA, 90 bp, Artificial sequence
  Description of sequence ADTB9 antigen Hu. CD45
  misc_feature (35)..(44): n is a, c, g, or t
  misc_feature (60)..(68): n is a, c, g, or t
gtgactggagttcagacgtgtgctcttccgatctnnnnnnnnnntcccttgcgatttacnnnnnnnnngctttaaggccggtcctagcaa

SEQ ID NO: 22 - DNA, 90 bp, Artificial sequence
  Description of sequence ADTB10 antigen Hu. ZAP70
  misc_feature (35)..(44): n is a, c, g, or t
  misc_feature (60)..(68): n is a, c, g, or t
gtgactggagttcagacgtgtgctcttccgatctnnnnnnnnnncttgcctgcaatcacnnnnnnnnngctttaaggccggtcctagcaa

SEQ ID NO: 23 - DNA, 90 bp, Artificial sequence
  Description of sequence ADTB11 antigen Hu. Perforin
  misc_feature (35)..(44): n is a, c, g, or t
  misc_feature (60)..(68): n is a, c, g, or t
gtgactggagttcagacgtgtgctcttccgatctnnnnnnnnnntcttcgtttgaacggnnnnnnnnngctttaaggccggtcctagcaa

SEQ ID NO: 24 - DNA, 90 bp, Artificial sequence
  Description of sequence ADTB12 antigen Hu. GrzmB
  misc_feature (35)..(44): n is a, c, g, or t
  misc_feature (60)..(68): n is a, c, g, or t
gtgactggagttcagacgtgtgctcttccgatctnnnnnnnnnngcgtgttgtggtattnnnnnnnnngctttaaggccggtcctagcaa

SEQ ID NO: 25 - DNA, 44 bp, Artificial sequence
  Description of sequence ADT B1; Antigen Hu. CD19; component 5′
  misc_feature (35)..(44): n is a, c, g, or t
gtgactggagttcagacgtgtgctcttccgatctnnnnnnnnnn

SEQ ID NO: 26 - DNA, 15 bp, Artificial sequence
  Description of sequence ADT B1; Antigen Hu. CD19; component barcode
ctgggcaattactcg

SEQ ID NO: 27 - DNA, 31 bp, Artificial sequence
  Description of sequence ADT B1; Antigen Hu. CD19; component 3′
  misc_feature (1)..(9): n is a, c, g, or t
nnnnnnnnngctttaaggccggtcctagcaa

SEQ ID NO: 28 - DNA, 44 bp, Artificial sequence; identical to SEQ ID NO: 25
  Description of sequence ADT B2; Antigen Hu. CD3; component 5′
  misc_feature (35)..(44): n is a, c, g, or t

SEQ ID NO: 29 - DNA, 15 bp, Artificial sequence
  Description of sequence ADT B2; Antigen Hu. CD3; component barcode
ctcattgtaactcct

SEQ ID NO: 30 - DNA, 31 bp, Artificial sequence; identical to SEQ ID NO: 27
  Description of sequence ADT B2; Antigen Hu. CD3; component 3′
  misc_feature (1)..(9): n is a, c, g, or t

SEQ ID NO: 31 - DNA, 44 bp, Artificial sequence; identical to SEQ ID NO: 25
  Description of sequence ADT B3; Antigen Hu. CD16; component 5′
  misc_feature (35)..(44): n is a, c, g, or t

SEQ ID NO: 32 - DNA, 15 bp, Artificial sequence
  Description of sequence ADT B3; Antigen Hu. CD16; component barcode
aagttcactctttgc

SEQ ID NO: 33 - DNA, 31 bp, Artificial sequence; identical to SEQ ID NO: 27
  Description of sequence ADT B3; Antigen Hu. CD16; component 3′
  misc_feature (1)..(9): n is a, c, g, or t

SEQ ID NO: 34 - DNA, 44 bp, Artificial sequence; identical to SEQ ID NO: 25
  Description of sequence ADT B4; Antigen Hu. CD4; component 5′
  misc_feature (35)..(44): n is a, c, g, or t

SEQ ID NO: 35 - DNA, 15 bp, Artificial sequence
  Description of sequence ADT B4; Antigen Hu. CD4; component barcode
tgttcccgctcaact

SEQ ID NO: 36 - DNA, 31 bp, Artificial sequence; identical to SEQ ID NO: 27
  Description of sequence ADT B4; Antigen Hu. CD4; component 3′
  misc_feature (1)..(9): n is a, c, g, or t

SEQ ID NO: 37 - DNA, 44 bp, Artificial sequence; identical to SEQ ID NO: 25
  Description of sequence ADT B5; Antigen Hu. CD11c; component 5′
  misc_feature (35)..(44): n is a, c, g, or t

SEQ ID NO: 38 - DNA, 15 bp, Artificial sequence
  Description of sequence ADT B5; Antigen Hu. CD11c; component barcode
tacgcctataacttg

SEQ ID NO: 39 - DNA, 31 bp, Artificial sequence; identical to SEQ ID NO: 27
  Description of sequence ADT B5; Antigen Hu. CD11c; component 3′
  misc_feature (1)..(9): n is a, c, g, or t

SEQ ID NO: 40 - DNA, 44 bp, Artificial sequence; identical to SEQ ID NO: 25
  Description of sequence ADT B6; Antigen Hu. CD56; component 5′
  misc_feature (35)..(44): n is a, c, g, or t

SEQ ID NO: 41 - DNA, 15 bp, Artificial sequence
  Description of sequence ADT B6; Antigen Hu. CD56; component barcode
tcctttcctgatagg

SEQ ID NO: 42 - DNA, 31 bp, Artificial sequence; identical to SEQ ID NO: 27
  Description of sequence ADT B6; Antigen Hu. CD56; component 3′
  misc_feature (1)..(9): n is a, c, g, or t

SEQ ID NO: 43 - DNA, 44 bp, Artificial sequence; identical to SEQ ID NO: 25
  Description of sequence ADT B7; Antigen Hu. CD14; component 5′
  misc_feature (35)..(44): n is a, c, g, or t

SEQ ID NO: 44 - DNA, 15 bp, Artificial sequence
  Description of sequence ADT B7; Antigen Hu. CD14; component barcode
tctcagacctccgta

SEQ ID NO: 45 - DNA, 31 bp, Artificial sequence; identical to SEQ ID NO: 27
  Description of sequence ADT B7; Antigen Hu. CD14; component 3′
  misc_feature (1)..(9): n is a, c, g, or t

SEQ ID NO: 46 - DNA, 44 bp, Artificial sequence; identical to SEQ ID NO: 25
  Description of sequence ADT B8; Antigen Hu. CD8; component 5′
  misc_feature (35)..(44): n is a, c, g, or t

SEQ ID NO: 47 - DNA, 15 bp, Artificial sequence
  Description of sequence ADT B8; Antigen Hu. CD8; component barcode
gcgcaacttgatgat

SEQ ID NO: 48 - DNA, 31 bp, Artificial sequence; identical to SEQ ID NO: 27
  Description of sequence ADT B8; Antigen Hu. CD8; component 3′
  misc_feature (1)..(9): n is a, c, g, or t

SEQ ID NO: 49 - DNA, 44 bp, Artificial sequence; identical to SEQ ID NO: 25
  Description of sequence ADT B9; Antigen Hu. CD45; component 5′
  misc_feature (35)..(44): n is a, c, g, or t

SEQ ID NO: 50 - DNA, 15 bp, Artificial sequence
  Description of sequence ADT B9; Antigen Hu. CD45; component barcode
tcccttgcgatttac

SEQ ID NO: 51 - DNA, 31 bp, Artificial sequence; identical to SEQ ID NO: 27
  Description of sequence ADT B9; Antigen Hu. CD45; component 3′
  misc_feature (1)..(9): n is a, c, g, or t

SEQ ID NO: 52 - DNA, 44 bp, Artificial sequence; identical to SEQ ID NO: 25
  Description of sequence ADT B10; Antigen Hu. ZAP70; component 5′
  misc_feature (35)..(44): n is a, c, g, or t

SEQ ID NO: 53 - DNA, 15 bp, Artificial sequence
  Description of sequence ADT B10; Antigen Hu. ZAP70; component barcode
cttgcctgcaatcac

SEQ ID NO: 54 - DNA, 31 bp, Artificial sequence; identical to SEQ ID NO: 27
  Description of sequence ADT B10; Antigen Hu. ZAP70; component 3′
  misc_feature (1)..(9): n is a, c, g, or t

SEQ ID NO: 55 - DNA, 44 bp, Artificial sequence; identical to SEQ ID NO: 25
  Description of sequence ADT B11; Antigen Hu. Perforin; component 5′
  misc_feature (35)..(44): n is a, c, g, or t

SEQ ID NO: 56 - DNA, 15 bp, Artificial sequence
  Description of sequence ADT B11; Antigen Hu. Perforin; component barcode
tcttcgtttgaacgg

SEQ ID NO: 57 - DNA, 31 bp, Artificial sequence; identical to SEQ ID NO: 27
  Description of sequence ADT B11; Antigen Hu. Perforin; component 3′
  misc_feature (1)..(9): n is a, c, g, or t

SEQ ID NO: 58 - DNA, 44 bp, Artificial sequence; identical to SEQ ID NO: 25
  Description of sequence ADT B12; Antigen Hu. GrzmB; component 5′
  misc_feature (35)..(44): n is a, c, g, or t

SEQ ID NO: 59 - DNA, 15 bp, Artificial sequence
  Description of sequence ADT B12; Antigen Hu. GrzmB; component barcode
gcgtgttgtggtatt

SEQ ID NO: 60 - DNA, 31 bp, Artificial sequence; identical to SEQ ID NO: 27
  Description of sequence ADT B12; Antigen Hu. GrzmB; component 3′
  misc_feature (1)..(9): n is a, c, g, or t

(19) United States
(12) Patent Application Publication
(10) Pub. No.: US 2023/0193246 A1
(43) Pub. Date: Jun. 22, 2023

(54) METHODS AND COMPOSITIONS FOR DETECTING TARGETS

(71) Applicant: BIOLEGEND, Inc., San Diego, CA (US)

(72) Inventors: Andre Luiz Viera Zorzetto Fernandes, San Diego, CA (US); Craig Monell, San Diego, CA (US); Kristopher Nazor, San Diego, CA (US); David Soper, San Diego, CA (US); Carsten Wiethe, San Diego, CA (US); Xifeng Yang, San Diego, CA (US); Bertrand Yeung, San Diego, CA (US)

(73) Assignee: BIOLEGEND, INC., San Diego, CA (US)

(21) Appl. No.: 17/908,821

(22) PCT Filed: Feb. 25, 2021
(86) PCT No.: PCT/US2021/019596

§ 371 Date: Sep. 1, 2022

Related U.S. Application Data
(60) Provisional application No. 62/985,849, filed on Mar. 5, 2020.

(51) Int. Cl. C12N 15/10

(52) U.S. Cl. CPC C12N 15/1065

(57) ABSTRACT

The disclosure relates in part to methods and compositions for detecting targets in a sample. Such methods and compositions can be useful for laboratory, research, and diagnostic purposes.

### CROSS-REFERENCES TO RELATED APPLICATIONS

This application claims priority to U.S. Application No. 62/985,849, filed Mar. 5, 2020, the contents of which is hereby incorporated by reference in its entirety for all purposes.

### FIELD OF THE DISCLOSURE

The technology relates in part to methods and compositions for detecting targets in a sample. In certain aspects, the technology relates in part to methods and compositions for detecting targets (e.g., biological targets, chemical targets) in a biological sample. Such methods and compositions can be useful for laboratory, research, and diagnostic purposes.

### BACKGROUND OF THE DISCLOSURE

The following description includes information that may be useful in understanding the present technology. It is not an admission that any of the information provided herein, or any publication specifically or implicitly referenced herein, is prior art, or even particularly relevant, to the presently claimed technology.

Cellular heterogeneity can be measured in several different ways, most commonly via genomic, epigenomic, transcriptomic, and proteomic studies. However, the level of heterogeneity at one level of expression or regulation may not be the same at another level. There are many causes of cellular heterogeneity. Firstly, populations of cells will naturally contain cells that develop random mutations. These unique subclones can become significant portions of the population if that mutation confers a selective advantage and proliferates. However, not all cellular heterogeneity is genetic. Rather, much heterogeneity is phenotypic, and is frequently expressed in transcriptomes that vary from cell to cell. Heterogeneity may be extrinsic or intrinsic, the former leading to phenotypic plasticity in response to an environmental change and the latter being a result of stochastic events (e.g., gene expression noise). The fluctuation in gene expression lends itself to varying levels of protein abundance in different cells within a population at a given time, which is most readily visualized using flow cytometry.

Flow cytometry is a quasi-quantitative technique useful for simultaneous detection of several biomarkers in a single cell. This technique allows for researchers to identify and group related cells within a population. While flow cytometry has been considered the gold standard for enumeration and/or characterization of different cell subsets within complex and heterogeneous populations, the technique provides very little information regarding intracellular distributions.

A new approach combines the benefits of flow cytometry with transcription profiling of individual cells, by simultaneously detecting extracellular protein markers and single-cell transcriptomes in a high-throughput fashion. Using ligands, barcoded with biopolymers, such as oligonucleotides or polypeptides, this method can convert protein detection into a quantitative readout. Current methods, however, are limited by the availability of cell surface markers, especially during disease states. There exists a need for a more sensitive and accurate characterization of subsets of cells within diseased populations. The present disclosure satisfies this and other needs and provides other advantages as well.

### BRIEF SUMMARY

Provided herein, in some aspects, are hybridization buffer compositions.

Also provided herein, in some aspects, are methods for detecting one or more targets in a sample. The target may be a biological target, or a chemical target. In some aspects, the methods comprise contacting the sample with one or more of: i) a composition comprising a first construct that comprises a first ligand attached or conjugated to a polymer construct by a linker, said first ligand binding specifically to a first target, and said polymer construct comprising: an amplification handle; a barcode that specifically identifies said first ligand; an optional unique molecular identifier that is positioned adjacent to the barcode on its 5′ or 3′ end; and an anchor for hybridizing to a capture sequence that comprises a sequence complementary to said anchor; ii) a composition comprising at least one additional construct, which construct comprises an additional ligand attached or conjugated to an additional polymer construct by a linker, said additional ligand binding specifically to an additional target, and said additional polymer construct comprising an amplification handle; an additional barcode that specifically identifies said additional ligand; an optional additional unique molecular identifier that is positioned adjacent to the additional barcode on its 5′ or 3′ end, and an anchor for hybridizing to a capture sequence that comprises a sequence complementary to said anchor; and/or iii) a composition comprising one or more substantially identical constructs, each substantially identical construct differing from any other reference first or additional construct in the sequence of its optional unique molecular identifier (UMI) or the absence of the UMI.

Additionally, provided herein, in some aspects are high throughput methods for detecting one or more targets in a sample, the method comprising contacting the sample with one or more of i) a composition comprising a first construct that comprises a first ligand that binds specifically to a first target, said first ligand attached or conjugated to a first polymer construct by a linker, where the first polymer construct comprises: an amplification handle; a barcode sequence that specifically identifies said first ligand from any other ligand that recognizes a different target, an optional unique molecular identifier sequence that is positioned adjacent to the 5′ or 3′ end of the barcode, and an anchor sequence for hybridizing to a capture sequence that comprises a sequence complementary to said anchor; ii) a composition of (i) comprising at least one additional construct, which comprises an additional ligand attached or conjugated to an additional polymer construct by a linker, said additional ligand binding specifically to an additional target, and said additional polymer construct comprising: an amplification handle; an additional barcode that specifically identifies said additional ligand; an optional additional unique molecular identifier that is positioned adjacent to the 5′ or 3′ end of the additional barcode, and an anchor sequence of (i), where said additional construct differs from any other construct in the composition in its ligand, target, barcode, and UMI; and/or iii) a composition of (i) or (ii) comprising one or more substantially identical constructs, each substantially identical construct differing from any other reference first or additional construct in the sequence of its optional unique molecular identifier (UMI) or the absence of the UMI.

In some aspects, provided herein, are high throughput methods for characterizing a cell by simultaneous detection of one or more targets located in or on the cell and the transcriptome, genome, or epigenome, the method comprising contacting a sample (e.g., a biological sample) containing cells with one or more of: i) a composition that comprises a first construct that comprises a first ligand that binds specifically to a first target located in or on the surface of a cell, said first ligand conjugated to a first polymer construct by a linker, where the first polymer construct comprises an amplification handle; a barcode sequence that specifically identifies said first ligand from any other ligand that recognizes a different target, an optional unique molecular identifier sequence that is positioned adjacent the 5′ or 3′ end of the barcode, and a polyA anchor sequence designed for hybridizing to a capture oligonucleotide sequence comprising a polyT sequence immobilized on a microfluidics bead, a slide, a microwell, or a nanowell; ii) a composition of (i) comprising at least one additional construct, which comprises an additional ligand conjugated to an additional polymer construct by a linker, said additional ligand binding specifically to an additional target, and said additional polymer construct comprising: the amplification handle of (i); an additional barcode that specifically identifies said additional ligand; an optional additional unique molecular identifier that is positioned adjacent the 5′ or 3′ end of the additional barcode, and the said anchor of (i), where said additional ligand, additional barcode, additional UMI and additional target differ from the corresponding components in any other construct in the composition; and/or iii) a composition of (i) or (ii) comprising one or more substantially identical constructs, each substantially identical construct differing from any other reference first or additional construct in the sequence of its optional unique molecular identifier (UMI) or the absence of the UMI.

Certain embodiments are described further in the following description, examples, claims and drawings.

### BRIEF DESCRIPTION OF THE DRAWINGS

The drawings illustrate certain embodiments of the technology and are not limiting.

For clarity and ease of illustration, the drawings are not made to scale and, in some instances, various aspects may be shown exaggerated or enlarged to facilitate an understanding of particular embodiments.

FIG. 1, panels A-I, show scatter plots showing the results of experiments described herein. Specifically, panels A-I show flow cytometry and next generation sequencing (NGS) side-by-side comparison for Perforin (dG9) stain. PBMC were surface stained with anti-CD8 (Brilliant Violet 421™ (BV421) format or tyramide signal amplification (TsA) format), anti-CD56 (phycoerythrin (PE) format or TsA format) and anti-CD19 (PerCP-Cy5.5 format or TsA format); fixed/permeabilized with BIOLEGEND FoxP3 Fix/Perm Buffer (Cat. #421401) and further stained with anti-Perforin (dG9) directly conjugated with ALEXA FLUOR 647, or anti-Perforin TsA format pre-incubated with oligo-dT-ALEXA FLUOR 647. Top row: Perforin detection on CD8 cells (A), NK cells (B) and B cells (C) using direct conjugate stained cells analyzed by flow cytometry; Middle row: Perforin detection on CD8 cells (D), NK cells (E) and B cells (F) using anti-Perforin TsA format pre-incubated with oligo-dT-ALEXA FLUOR 647 stained cells analyzed by flow cytometry; Bottom row: Perforin detection on CD8 cells (G), NK cells (H) and B cells (I) using anti-Perforin TsA stained cells analyzed by NGS.

FIG. 2 shows electrophoresis of antibody-derived tags (ADTs) after amplification and indexing. Lane 1, ladder; Lane 2, methanol fixation and permeabilization; Lane 3, paraformaldehyde (1%) fixation and methanol (80%) permeabilization; Lane 4, paraformaldehyde (1%) fixation and saponin permeabilization; Lane 5, paraformaldehyde (1%) fixation and TWEEN 20 permeabilization; Lane 6, paraformaldehyde (1%) fixation and TRITON X-100 permeabilization; and Lane 7, paraformaldehyde (1%) fixation and NP40 permeabilization.

FIG. 3 shows electrophoresis of cDNA isolated from PBMCs. Lane 1, ladder; Lane 2, PBMCs without fixation; Lane 3, paraformaldehyde (4%) fixation; Lane 4, paraformaldehyde (1%) fixation; Lane 5, methanol (80%) fixation; Lane 6, methanol (80%) fixation; Lane 7, paraformaldehyde (1%) fixation and methanol (100%) permeabilization; Lane 8, paraformaldehyde (1%) fixation and saponin permeabilization; Lane 9, paraformaldehyde (1%) fixation and TWEEN 20 permeabilization; Lane 10, paraformaldehyde (1%) fixation and TRITON X-100 permeabilization; Lane 11, paraformaldehyde (1%) fixation and NP40 permeabilization.

FIG. 4, panels A-I, shows Uniform Manifold Approximation and Projection (UMAP) plots of PBMC populations. Cells were clustered based on expression of CD45 (panel A), CD3 (panel B), CD8 (panel C), CD4 (panel D), CD56 (panel E), CD16 (panel F), CD19 (panel G), CD11c (panel H), or CD14 (panel I).

FIG. 5, panels A-J, shows UMAP plots of clustered cells based on antibody-derived tags (ADTs) and cDNA (CD3, CD8, CD56, perforin, and Zap70).

FIG. 6, panels A-J, shows UMAP plots of clustered cells based on antibody-derived tags (ADTs).

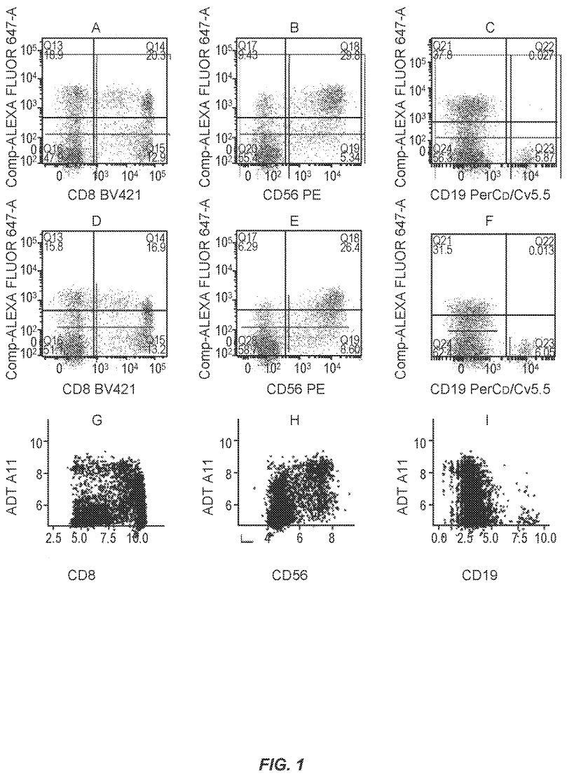

### DETAILED DESCRIPTION OF THE DISCLOSURE

Methods and compositions for detecting targets in a sample (e.g., biological sample) may be useful for certain research and clinical applications, as well as diagnostics. Provided herein are methods for detecting targets in a sample (e.g., biological sample) using ligand-polymer conjugates, which increase the sensitivity of a variety of assay methodologies, methods and compositions for detecting intracellular targets in a sample (e.g., biological sample), and buffers for use in methods for detecting intracellular targets. Use of the methods and compositions provided herein is highly scalable, limited only by the number of specific ligands, e.g., antibodies, that are available. Also provided herein are methods and compositions for spatial and bulk detection of nucleic acid and protein in a biological sample, e.g., organelles, exosomes, cells, cellular lysates, tissues, blood, serum, plasma, and saliva. The methods and compositions described herein which employ molecular barcoding of ligands allow multiplexing to virtually any number of parameters, more specifically, the methods and compositions disclosed herein allow multiplexing using intracellular targets.

### Compositions

The compositions described herein comprise one or more of the constructs, first constructs and additional constructs, a variety of selection of construct components as described herein, and buffers.

Buffers

Hybridization Buffer

The disclosed hybridization buffer compositions include at least a pH buffer solution, e.g., saline-sodium citrate (SSC) or phosphate buffered saline (PBS), bovine serum albumin (BSA), a reducing agent, e.g., dithiothreitol (DTT), and a blocking agent (e.g., biopolymer, e.g., oligonucleotide or polypeptide; single-stranded binding proteins). This combination of components provides a hybridization composition that can be used as a binding reagent. In some embodiments, the hybridization buffer may be useful in assays that require the labeling of biomolecules. In some embodiments, the hybridization buffer may be useful in assays that require the labeling of nucleic acids. In some embodiments, the hybridization buffer may be useful in assays that require the labeling of genomic DNA and RNA. In some embodiments, the buffer may be useful in assays that require the labeling of mitochondrial DNA. In some embodiments, the hybridization buffer may be useful in assays that require the labeling of proteins. In some embodiments, the buffer may be useful in assays that require the labeling of post-translational modifications. In some embodiments, the buffer may be useful in assays that require the labeling of non-protein antigens. In some embodiments, the buffer may be useful in assays that require the labeling of metabolites. In preferred embodiments, the hybridization buffer may be useful in assays that require the labeling of intracellular proteins. In some embodiments, the hybridization buffer may be useful in conjunction with CITE-seq, REAP-seq, and AbSeq methodologies. In some embodiments, the buffer may be useful in conjunction with microscopy based spatial profiling methodologies. In some embodiments, the buffer may be useful in conjunction with next generation sequencing based spatial profiling methodologies. In some embodiments, the hybridization buffer composition may inhibit non-specific binding of construct compositions to biomolecules.

Examples of pH buffer solutions include citrate buffers (saline-sodium citrate (SSC)), phosphate buffers (phosphate buffered saline (PBS)), tris (tris(hydroxymethyl)aminomethane) or (2-amino-2-(hydroxymethyl)propane-1,3-diol)-based buffers, TAPS ([tris(hydroxymethyl)methylamino]propanesulfonic acid)-based buffers, HEPES (4-(2-hydroxyethyl)-1-piperazineethanesulfonic acid)-based buffers, Bicine (2-(bis(2-hydroxyethyl)amino)acetic acid)-based buffers, tricine (N-[tris(hydroxymethyl)methyl]glycine)-based buffers, TAPSO (3-[N-tris(hydroxymethyl)methylamino]-2-hydroxypropanesulfonic acid)-based buffers, TES (2-[[1,3-dihydroxy-2-(hydroxymethyl)propan-2-yl]amino]ethanesulfonic acid)-based buffers, MES (2-(N-morpholino)ethanesulfonic acid)-based buffers, PIPES (piperazine-N,N′-bis(2-ethanesulfonic acid))-based buffers, Cacodylate (dimethylarsenic acid)-based buffers, and the like.

In some embodiments, the oligonucleotides are phosphorothioate (PS) oligonucleotides. In some embodiments, the oligonucleotides comprise at least about 5 nucleotides to 150 nucleotides, at least about 10 nucleotides to 140 nucleotides, at least about 20 nucleotides to 130 nucleotides, at least about 30 nucleotides to 120 nucleotides, at least about 40 nucleotides to 110 nucleotides, at least about 50 nucleotides to 100 nucleotides, at least about 60 nucleotides to 90 nucleotides, at least about 70 nucleotides to 80 nucleotides. In some embodiments, the oligonucleotides comprise different nucleotides. In some embodiments, the oligonucleotides comprise the same repeating nucleotide. In some embodiments, the oligonucleotide is a random sequence of nucleotides. In some embodiments, the oligonucleotide is a complementary sequence of nucleotides. In some embodiments, the oligonucleotide is conjugated to an antibody.

In some embodiments, the hybridization buffer comprises heparin. In some embodiments, the hybridization buffer comprises salmon sperm. In some embodiments, the hybridization buffer comprises herring sperm. In some embodiments, the hybridization buffer comprises human serum. In some embodiments, the hybridization buffer comprises bovine serum albumin. In some embodiments, the hybridization buffer comprises fetal bovine serum. In some embodiments, the hybridization buffer comprises knock-out serum. In some embodiments, the hybridization buffer comprises serum from an autologous donor.

Fixation Buffer

The disclosed fixation buffer compositions include at least a fixative, e.g., methanol (e.g., about 40% to about 100% methanol) or paraformaldehyde (PFA), a pH buffer solution, e.g., saline-sodium citrate (SSC) or phosphate buffered solution (PBS), bovine serum albumin (BSA), a reducing agent, e.g., dithiothreitol (DTT), and an enzyme inhibitor, e.g., a nuclease, protease, and/or protease inhibitor. This combination of components provides a buffer composition that can be used as a fixing reagent. In some embodiments, the fixation buffer may be useful in assays that require the labeling of or useful for intracellular staining of biomolecules. In some embodiments, fixation buffer compositions are used to fix cells prior to permeabilization. In some embodiments, fixation buffer compositions are used to fix cells at the same time as permeabilization. In some embodiments, the buffer may be useful in assays that require the labeling of biomolecules. In some embodiments, the buffer may be useful in assays that require the labeling of nucleic acids. In some embodiments, the buffer may be useful in assays that require the labeling of genomic DNA. In some embodiments, the buffer may be useful in assays that require the labeling of mitochondrial DNA. In some embodiments, the buffer may be useful in assays that require the labeling of RNA. In some embodiments, the buffer may be useful in assays that require the labeling of proteins. In some embodiments, the buffer may be useful in assays that require the labeling of post-translational modifications. In some embodiments, the buffer may be useful in assays that require the labeling of non-protein antigens. In some embodiments, the buffer may be useful in assays that require the labeling of metabolites. In preferred embodiments, the buffer may be useful in assays that require the labeling of intracellular proteins. In some embodiments, the buffer may be useful in conjunction with CITE-seq, REAP-seq, and AbSeq methodologies. In some embodiments, the buffer may be useful in conjunction with microscopy based spatial profiling methodologies. In some embodiments, the buffer may be useful in conjunction with next generation sequencing based spatial profiling methodologies.

In some embodiments, the fixation buffer may comprise formalin. In other embodiments, the fixation buffer may comprise ethanol. In some embodiments, the fixation buffer may comprise an RNAse inhibitor. In some embodiments, the fixation buffer may comprise a protease inhibitor.

Examples of pH buffer solutions include citrate buffers (saline-sodium citrate (SSC)), phosphate buffers (phosphate buffered saline (PBS)), tris (tris(hydroxymethyl)aminomethane) or (2-amino-2-(hydroxymethyl)propane-1,3-diol)-based buffers, TAPS ([tris(hydroxymethyl)methylamino]propanesulfonic acid)-based buffers, HEPES (4-(2-hydroxyethyl)-1-piperazineethanesulfonic acid)-based buffers, Bicine (2-(bis(2-hydroxyethyl)amino)acetic acid)-based buffers, tricine (N-[tris(hydroxymethyl)methyl]glycine)-based buffers, TAPSO (3-[N-tris(hydroxymethyl)methylamino]-2-hydroxypropanesulfonic acid)-based buffers, TES (2-[[1,3-dihydroxy-2-(hydroxymethyl)propan-2-yl]amino]ethanesulfonic acid)-based buffers, MES (2-(N-morpholino)ethanesulfonic acid)-based buffers, PIPES (piperazine-N,N′-bis(2-ethanesulfonic acid))-based buffers, Cacodylate (dimethylarsenic acid)-based buffers, and the like.

In some embodiments, the fixation buffer may comprise sodium saline citrate (SSC), 4-(2-hydroxyethyl)-1-piperazineethanesulfonic acid (HEPES), SSPE, piperazine-N,N′-bis(2-ethanesulfonic acid) (PIPES), tetramethyl ammonium chloride (TMAC), Tris(hydroxymethyl)aminomethane (Tris), SET (sucrose/EDTA/Tris), citric acid, potassium phosphate or sodium pyrophosphate. In some embodiments, the fixation buffer may comprise SSC. SSC is a buffering agent used to maintain the pH of a solution near a chosen value after the addition of another acid or base. In some embodiments, the fixation buffer may comprise about 0.5×SSC, about 1×SSC, about 1.5×SSC, about 2×SSC, about 2.5×SSC, about 3×SSC, about 3.5×SSC, about 4×SSC, about 4.5×SSC, or about 5×SSC. In some embodiments, the fixation buffer may comprise 3×SSC. In some embodiments, the fixation buffer may comprise PBS. In some embodiments, the fixation buffer may comprise about 2×PBS, about 2.5×PBS, about 3×PBS, about 3.5×PBS, about 4×PBS, about 4.5×PBS, or about 5×PBS. In some embodiments, the fixation buffer may comprise 3×PBS.

In some embodiments, the fixation buffer may comprise bovine serum albumin (BSA). In some embodiments, the fixation buffer may comprise about 1% (w/v) to about 20% (w/v) BSA, about 2% (w/v) to about 19% (w/v) BSA, about 3% (w/v) to about 18% (w/v) BSA, about 4% (w/v) to about 17% (w/v) BSA, about 5% (w/v) to about 16% BSA, about 6% (w/v) to about 15% (w/v) BSA, about 7% (w/v) to about 14% (w/v) BSA, about 8% (w/v) to about 13% (w/v) BSA, about 9% (w/v) to about 12% (w/v) BSA, or about 10% (w/v) to about 11% (w/v) BSA. In some embodiments, the fixation buffer may comprise 1% (w/v) BSA.

In some embodiments, the fixation buffer may comprise dithiothreitol (DTT). In some embodiments, the fixation buffer may comprise about 1 mM to about 10 mM DTT, about 2 mM to about 9 mM DTT, about 3 mM to about 8 mM DTT, about 4 mM to about 7 mM DTT, or about 5 mM to about 6 mM DTT. In some embodiments, the fixation buffer may comprise 1 mM DTT.

In some embodiments, the fixation buffer may comprise an RNAse inhibitor. In some embodiments, the RNAse inhibitor may be a protein-based inhibitor. In some embodiments, the fixation buffer may comprise about 1% (w/v) to about 10% (w/v) RNAse inhibitor, about 2% (w/v) to about 9% (w/v) RNAse inhibitor, about 3% (w/v) to about 8% (w/v) RNAse inhibitor, about 4% (w/v) to about 7% (w/v) RNAse inhibitor, about 5% (w/v) to about 6% (w/v) RNAse inhibitor. In some embodiments, the fixation buffer may comprise 1% RNAse inhibitor.

In some embodiments, the fixation buffer may comprise paraformaldehyde (PFA). In some embodiments, the fixation buffer may comprise about 1% to about 10% PFA, about 2% to about 9% PFA, about 3% to about 8% PFA, about 4% to about 7% PFA, about 5% to about 6% PFA. In some embodiments, the fixation buffer may comprise 1% PFA.

Wash Buffer

The disclosed wash buffer compositions include at least a pH buffer solution, e.g., sodium saline citrate (SSC) or phosphate buffered saline (PBS), bovine serum albumin (BSA), and a reducing agent, e.g., dithiothreitol (DTT). This combination of components provides a buffer composition that can be used as a washing reagent. In some embodiments, the wash buffer may be useful in assays that require the labeling of or useful for intracellular staining of biomolecules. In some embodiments, wash buffer compositions are used to wash cells prior to fixation. In some embodiments, wash buffer compositions are used to wash cells after fixation. In some embodiments, wash buffer compositions are used to wash cells prior to permeabilization. In some embodiments, wash buffer compositions are used to wash cells following permeabilization. In some embodiments, wash buffer compositions are used to wash cells after staining. In some embodiments, the buffer may be useful in assays that require the labeling of nucleic acids. In some embodiments, the buffer may be useful in assays that require the labeling of genomic DNA. In some embodiments, the buffer may be useful in assays that require the labelling RNA. In some embodiments, the buffer may be useful in assays that require the labeling of mitochondrial DNA. In some embodiments, the buffer may be useful in assays that require the labeling of proteins. In some embodiments, the buffer may be useful in assays that require the labeling of post-translational modifications. In some embodiments, the buffer may be useful in assays that require the labeling of non-protein antigens. In some embodiments, the buffer may be useful in assays that require the labeling of metabolites. In preferred embodiments, the buffer may be useful in assays that require the labeling of intracellular proteins. In some embodiments, the buffer may be useful in conjunction with CITE-seq, REAP-seq, and AbSeq methodologies. In some embodiments, the buffer may be useful in conjunction with microscopy based spatial profiling methodologies. In some embodiments, the buffer may be useful in conjunction with next generation sequencing based spatial profiling methodologies.

Examples of pH buffer solutions include citrate buffers (saline-sodium citrate (SSC)), phosphate buffers (phosphate buffered saline (PBS)), tris (tris(hydroxymethyl)aminomethane) or (2-amino-2-(hydroxymethyl)propane-1,3-diol)-based buffers, TAPS ([tris(hydroxymethyl)methylamino]propanesulfonic acid)-based buffers, HEPES (4-(2-hydroxyethyl)-1-piperazineethanesulfonic acid)-based buffers, Bicine (2-(bis(2-hydroxyethyl)amino)acetic acid)-based buffers, tricine (N-[tris(hydroxymethyl)methyl]glycine)-based buffers, TAPSO (3-[N-tris(hydroxymethyl)methylamino]-2-hydroxypropanesulfonic acid)-based buffers, TES (2-[[1,3-dihydroxy-2-(hydroxymethyl)propan-2-yl]amino]ethanesulfonic acid)-based buffers, MES (2-(N-morpholino)ethanesulfonic acid)-based buffers, PIPES (piperazine-N,N′-bis(2-ethanesulfonic acid))-based buffers, Cacodylate (dimethylarsenic acid)-based buffers, and the like.

In some embodiments, the wash buffer may comprise sodium saline citrate (SSC). In some embodiments, the wash buffer may comprise about 0.5×SSC, about 1×SSC, about 1.5×SSC, about 2×SSC, about 2.5×SSC, or about 3×SSC. In some embodiments, the wash buffer may comprise 3×SSC.

In some embodiments, the wash buffer may comprise bovine serum albumin (BSA). In some embodiments, the wash buffer may comprise about 1% (w/v) to about 20% (w/v) BSA, about 2% (w/v) to about 19% (w/v) BSA, about 3% (w/v) to about 18% (w/v) BSA, about 4% (w/v) to about 17% (w/v) BSA, about 5% (w/v) to about 16% BSA, about 6% (w/v) to about 15% (w/v) BSA, about 7% (w/v) to about 14% (w/v) BSA, about 8% (w/v) to about 13% (w/v) BSA, about 9% (w/v) to about 12% (w/v) BSA, or about 10% (w/v) to about 11% (w/v) BSA. In some embodiments, the wash buffer may comprise 1% (w/v) BSA.

In some embodiments, the wash buffer may comprise dithiothreitol (DTT). In some embodiments, the wash buffer may comprise about 1 mM to about 10 mM DTT, about 2 mM to about 9 mM DTT, about 3 mM to about 8 mM DTT, about 4 mM to about 7 mM DTT, or about 5 mM to about 6 mM DTT. In some embodiments, the wash buffer may comprise 1 mM DTT.

In some embodiments, the wash buffer may comprise a detergent. In some embodiments, a detergent is present in the wash buffer at a concentration between about 0.01% to about 0.25%. For example, a detergent may present in the wash buffer at a concentration of about 0.05%, 0.1%, 0.15%, 0.2%, or 0.25%. In some embodiments, a detergent is present in the wash buffer at a concentration of about 0.5%. In some embodiments, the detergent may be saponin.

Permeabilization Buffer

The disclosed permeabilization buffer compositions may include one or more components chosen from sodium saline citrate (SSC), phosphate buffered saline (PBS), bovine serum albumin, reducing agent, e.g., dithiothreitol (DTT), enzyme inhibitor, nonionic surfactant, polyoxyethylene sorbitol ester, ethoxylated nonylphenol, saponin, and an alcohol, e.g., methanol or ethanol. This combination of components provides a permeabilization composition that can be used as a permeabilization reagent. In some embodiments, the permeabilization buffer may be useful in assays that require the labeling of or useful for intracellular staining of biomolecules. In some embodiments, permeabilization buffer compositions are used to permeabilize cells prior to staining. In some embodiments, the buffer may be useful in assays that require the labeling of biomolecules. In some embodiments, the buffer may be useful in assays that require the labeling of nucleic acids. In some embodiments, the buffer may be useful in assays that require the labeling of genomic DNA. In some embodiments, the buffer may be useful in assays that require the labeling of mitochondrial DNA. In some embodiments, the buffer may be useful in assays that require the labeling of RNA. In some embodiments, the buffer may be useful in assays that require the labeling of proteins. In some embodiments, the buffer may be useful in assays that require the labeling of post-translational modifications. In some embodiments, the buffer may be useful in assays that require the labeling of non-protein antigens. In some embodiments, the buffer may be useful in assays that require the labeling of metabolites. In preferred embodiments, the buffer may be useful in assays that require the labeling of intracellular proteins. In some embodiments, the buffer may be useful in conjunction with CITE-seq, REAP-seq, and AbSeq methodologies. In some embodiments, the buffer may be useful in conjunction with microscopy based spatial profiling methodologies. In some embodiments, the buffer may be useful in conjunction with next generation sequencing based spatial profiling methodologies.

In some embodiments, the permeabilization buffer comprises a detergent. Detergents may include, for example, saponin, TRITON X-100, TWEEN 20, NP-40, and the like. In some embodiments, a detergent is present in the permeabilization buffer at a concentration between about 0.01% (w/v) to about 0.25% (w/v). For example, a detergent may be present in the permeabilization buffer at a concentration of about 0.05% (w/v), 0.1% (w/v), 0.15% (w/v), 0.2% (w/v), or 0.25% (w/v). In some embodiments, a detergent is present in the permeabilization buffer at a concentration of about 0.5% (w/v).

In some embodiments, the permeabilization buffer comprises an alcohol. Alcohols may include, for example, methanol, ethanol, and the like. In some embodiments, an alcohol is present in the permeabilization buffer at a concentration between about 40% (v/v) to about 100% (v/v). For example, an alcohol may be present in the permeabilization buffer at a concentration of about 40% (v/v), 50% (v/v), 60% (v/v), 70% (v/v), 80% (v/v), 90% (v/v), or 100% (v/v). In some embodiments, an alcohol is present in the permeabilization buffer at a concentration of about 80% (v/v).

In some embodiments, the permeabilization buffer may comprise a nuclease inhibitor. In some embodiments, the permeabilization buffer may comprise a nuclease inhibitor. In some embodiments, the permeabilization buffer may comprise an RNAse inhibitor. In some embodiments, the permeabilization buffer may comprise a protease inhibitor. In some embodiments, the permeabilization buffer may comprise a phosphatase inhibitor.

Constructs

The selection of the components of the composition will depend upon the identity of the target sought, the next generation sequencing and amplification protocols employed and the purpose of the assay method. In the methods section below, the exemplified methods employ Drop-seq methodologies; however, other methods may be used. The method used may dictate the selection and compositions of the various components described herein which make up the composition. Thus the following description of compositions is not exhaustive, and one of skill in the art can design many different compositions based on the teachings provided herein. The composition may also contain the constructs in a suitable carrier or excipient. The elements of each composition will depend upon the assay format in which it will be employed.

In some embodiments, a composition comprises a “first” construct that comprises a “first” ligand attached or conjugated to a polymer construct, e.g., a construct oligonucleotide sequence, by a linker. In these embodiments, the construct oligonucleotide sequence comprises a) an amplification handle; b) a barcode that specifically identifies the first ligand; c) an optional unique molecular identifier that is positioned adjacent to the barcode on its 5′ or 3′ end; and d) an anchor (e.g., of at least 3 nucleotides) for hybridizing to a capture oligonucleotide sequence that comprises a sequence complementary to the anchor. In some embodiments, the first ligand binds specifically to a first target located in or on the surface of a cell, such as a cell surface antigen or epitope.

In other embodiments, a composition comprises multiple substantially identical “first” constructs, where each substantially identical first construct differs from the reference “first” construct only in the sequence of the optional unique molecular identifier or its absence from the construct. Yet other embodiments of the composition include at least one additional construct, which comprises an additional ligand attached or conjugated to an additional construct oligonucleotide sequence by a linker, the additional ligand binding specifically to an additional target located in or on the surface of a cell, and the additional construct oligonucleotide sequence comprising: a) an amplification handle; b) an additional barcode that specifically identifies the additional ligand; c) an optional additional unique molecular identifier that is positioned adjacent to the additional barcode on its 5′ or 3′ end, and d) an anchor of at least 3 nucleotides for hybridizing to a complementary sequence. In some embodiments, the amplification handle or anchor also differ from the corresponding components in any other construct in the composition. The components specifically identified as “additional” components differ from the corresponding components in any other construct in the composition. In other embodiments, a composition comprises multiple substantially identical “additional” constructs, where each substantially identical additional construct differs from the reference “additional” construct only in the sequence of the optional unique molecular identifier or its absence from the construct. The number of constructs in a single composition is limited only by the number of targets desired to be identified and/or quantified.

In some embodiments, in specific compositions, the first or additional ligand is an antibody or antibody fragment and the first or additional target is a cell surface epitope. In other specific compositions, the first or additional ligand is an antibody or antibody fragment and the first or additional target is an intracellular protein. Any number of compositions may be prepared with various combinations of ligands and targets as discussed herein. For example, a cell hashtag construct preferably uses a ligand that targets a broadly expressed cellular protein, based on the differences in intended use of these constructs in contrast to the CITE-seq constructs, as described herein.

In another composition, the first construct comprises a first ligand (e.g., a first antibody or fragment thereof) attached or conjugated to a construct oligonucleotide sequence by a linker, the first ligand (e.g., the first antibody or fragment thereof) binding specifically to a first target (e.g., first epitope sequence) located on the surface of a cell, and the construct oligonucleotide sequence comprising: an amplification handle; a barcode that specifically identifies the first ligand (e.g., a first antibody or fragment thereof); an optional unique molecular identifier that is positioned adjacent to the barcode on its 5′ or 3′ end; and a polyA anchor sequence of at least 3 nucleotides for hybridizing to a polyT sequence. This type of composition is particularly suitable where the complementary polyT sequence is immobilized on a substrate, e.g., a microfluidics bead, a slide, a microwell, or a nanowell. As described, this composition's construct contains a linker that comprises biotin, which is bound to the 5′ end of the construct oligonucleotide sequence by a disulfide bond; and streptavidin, which is fused to the antibody or antibody fragment. Other compositions can be designed containing multiple of these first constructs, which differ only in the sequence of the optional unique molecular identifier or its absence from the construct.

In yet further embodiments, the composition contains at least one additional construct, which comprises at least one additional ligand (e.g., one additional antibody or fragment thereof) that binds specifically to an additional target (e.g., additional epitope) located in or on the surface of a cell. The additional ligand (e.g., additional antibody or fragment thereof) is conjugated with an additional construct oligonucleotide sequence by a linker, where the additional construct oligonucleotide sequence comprises from 5′ to 3′: an amplification handle; an additional barcode sequence that specifically identifies the additional ligand (e.g., an additional antibody or fragment thereof) from any other ligand that recognizes an additional target (e.g., additional epitope), an optional additional unique molecular identifier sequence that is positioned adjacent to the barcode on its 5′ or 3′ end, and a polyA sequence of at least 3 nucleotides designed for hybridizing to a polyT sequence, where the additional components differ from the corresponding components in any other construct. In other embodiments, the amplification handle or anchor differ from the corresponding components in any other construct in the composition.

Other specific compositions contain an antibody mimetic as the first ligand and the first target is an intracellularly expressed protein that is present in a biological sample of biopsy tissue. The first construct comprises the antibody mimetic designed for binding to the target protein covalently attached to a construct oligonucleotide sequence by a disulfide linker. The construct oligonucleotide sequence comprises in 5′ to 3′ order: an amplification handle; a barcode that specifically identifies the first antibody mimetic; a UMI is positioned adjacent to the barcode on its 5′ end; and a polyA anchor sequence. The compositions also contain one or more substantially identical first constructs, each substantially identical first construct differing from the reference “first construct” by containing a different sequence for the UMI. In some embodiments, a substantially identical construct contains no UMI.

In yet further embodiments, the composition contains two additional constructs. Each additional construct comprises a different antibody mimetic which specifically binds a different protein present in the biopsied tissue sample. Each of the two additional constructs comprises the antibody mimetic conjugated with its additional construct oligonucleotide sequence by a linker. Each linker can be an optional chemistry as taught herein. In one such additional construct, the construct oligonucleotide sequence comprises from 3′ to 5′: an amplification handle; a barcode sequence that specifically identifies the additional antibody mimetic from any other antibody or fragment that recognizes a different protein target from the first constructs, and an additional different UMI sequence that is positioned adjacent to the barcode on its 3′ end, and a polyA sequence of at least 5 nucleotides designed for hybridizing to a polyT sequence. In other embodiments, the second additional construct comprises from 5′ to 3′: an amplification handle; a barcode sequence that specifically identifies an antibody mimetic different from those of the first constructs and from the first additional construct, and which recognizes a third protein target different from the first construct or first additional construct. This second additional construct contains no UMI but contains a polyA sequence of at least 3 nucleotides designed for hybridizing to a polyT sequence. These two additional constructs have targets, antibody mimetic ligands, barcodes, and UMIs (if present) that differ from each other's corresponding components and differ from the corresponding components in the “first” construct and any substantially identical “first” constructs present in the composition. It should further be understood that compositions may also have one or more substantially identical additional constructs, which differ from the reference additional construct by the UMI, as described above.

Many other types of ligands, targets, samples, UMIs, and barcodes as described above can be used to generate a wide variety of compositions as described herein.

Kits

Kits containing the compositions are also provided. Such kits will contain one or more first or additional constructs, one or more preservatives, stabilizers, or buffers, and such suitable assay and amplification reagents depending upon the amplification and analysis methods and protocols with which the composition will be used. Still other components in a kit include optional reagents for cleavage of the linker, a wash buffer, a blocking solution, a lysis buffer, and an encapsulation solution, detectable labels, immobilization substrates, optional substrates for enzymatic labels, as well as other laboratory items.

### Methods for Detection

The compositions and kits described above can be used in diverse environments for detection of different targets, by employing any number of assays and methods for detection or targets in general.

In some embodiments, methods for detecting one or more targets in a sample (e.g., biological sample) use the buffers and compositions described herein. The method may include the steps of contacting the biological sample with one or more of the buffers and compositions described herein. In some embodiments, the sample is contacted with a composition comprising a first construct that has a first ligand attached or conjugated to a polymer construct, e.g., a construct oligonucleotide sequence, by a linker. In some embodiments, the first ligand binds specifically to a first target located in a cell or on the surface of a cell, such as a cell surface epitope. The construct oligonucleotide sequence comprises: an amplification handle; a barcode that specifically identifies the first ligand; an optional unique molecular identifier that is positioned adjacent to the barcode on its 5′ and/or 3′ end; and an anchor for hybridizing to a complementary sequence for generation of a double stranded oligonucleotide sequence. In some embodiments, the sample is contacted with a composition comprising substantially identical “first” constructs, where each substantially identical first construct differs from the reference “first” construct only in the sequence of the optional UMI or its absence from the construct. Therefore, the sample is contacted with multiple ligands to the same cell surface epitope target.

In still other embodiments of the method, the sample is contacted with a first construct as described above (or multiples thereof); and a composition comprising at least one additional construct. The additional ligand is covalently attached or conjugated to an additional construct oligonucleotide sequence by a linker, the additional ligand binding specifically to an additional target located in a cell or on the surface of a cell. Thus, the additional target is in one embodiment a different cell surface epitope. The additional construct oligonucleotide sequence comprising: an amplification handle; an additional barcode that specifically identifies the additional ligand; an optional additional unique molecular identifier that is positioned adjacent to the additional barcode on its 5′ and/or 3′ end, and an anchor of at least 3 nucleotides for hybridizing to a complementary sequence for generation of a double stranded oligonucleotide sequence, where the additional components differ from the corresponding components in any other construct in the composition. In yet other embodiments, the amplification handle or anchor differ from the corresponding components in any other construct in the composition. It should be understood, that in this embodiment any number of additional constructs can be designed as described above to bind as many cell epitopes as desired, limited only by the choice and number of ligands. As described herein, in other embodiments the composition may contain one or more substantially identical “additional” constructs, where each substantially identical additional construct differs from the reference “additional” construct only in the sequence of the optional UMI or its absence from the construct.

In such methods, following the contacting and binding that occurs between cells in the sample (e.g., biological sample) and the first ligand in the first construct and desired number of additional ligands in the additional construct(s), the sample is washed to remove unbound constructs, if any. For each construct bound to its target epitope, the anchor sequence is then hybridized to its corresponding capture oligo complementary sequence. This can occur by addition of primers as capture complementary sequences or a capture oligo complementary sequence immobilized on a substrate, such as a bead, a slide, a multi-well plate, a chip, a microwell, or a nanowell. In certain embodiments, the 5′ end of the complementary sequence further comprises: an additional amplification handle; an additional barcode that specifically identifies the substrate to which the capture oligo sequence is bound; and an optional additional unique molecular identifier that is positioned adjacent to the additional barcode on its 5′ and/or 3′ end that identifies each capture oligo sequence.

Once the capture oligo with its complementary sequence is present in the sample, generation of double stranded oligonucleotide sequences can occur. In certain embodiments, the methods also include optionally inserting one or more UMIs to a position adjacent to the barcode on its 5′ and/or 3′ end or at any other portion, provided that the insertion does not prevent the functions of the components of the construct oligonucleotide sequence before or after anchor hybridization.

The detection methods, in some embodiments, include detecting the construct barcode sequences from each first and additional construct to identify whether the sample (e.g., biological sample) expresses or contains the first target (e.g., epitope) the additional targets (e.g., one or multiple additional cell surface epitopes) or a combination of the first target and additional targets (e.g., multiple different epitopes).

In yet other embodiments of this detection method, the expression level of the first target or additional targets in the sample (e.g., biological sample) is determined by detecting the amount of the corresponding construct barcodes. In some embodiments, the detection is performed by normalization to the amount of any one of unique molecular identifiers or the mean amount of two or more of unique molecular identifiers.

Various embodiments of the methods can include adding to the sample (e.g., biological sample) the composition containing the first construct(s) only, or compositions containing additional construct(s) simultaneously or sequentially prior to the washing step. Further method steps can include isolating the sample into individual cells or populations of cells before the contacting step or after the washing step. Another step involves extending the capture oligonucleotide hybridized to the anchor sequence to copy the construct barcode, UMI and amplification handle onto double stranded sequences. The double stranded oligonucleotide sequences can also be generated after anchor hybridization with primers annealed to the amplification handles after either anchor hybridization or insertion of UMIs.

Other variations of these methods involve cleaving the ligand from the construct prior to or after anchor hybridization to the complementary sequence. Still other embodiments involve lysing the cell, when desired. In various embodiments, the lysis technique can involve exposure of the cells to detergents, detergent-buffer solutions, such as RIPA buffer, IP-lysis buffers, M-PER or B-PER reagent solutions (Pierce Chemical) and the like. The ligand-oligonucleotide constructs can be used with targets other than intracellular antibodies and ligands other than antibodies as discussed herein.

Further embodiments involve cell permeabilization and optional fixation procedures before the contacting step or between sequential contacting steps with first or additional constructs. In various embodiments, the permeabilizing technique can involve exposure of the samples (e.g., biological samples) to permeabilization buffer(s) disclosed herein. The fixation step is optional before or during the permeabilization. Techniques of fixation are known to one of skill in the art, for example contacting the samples with a fixation buffer disclosed herein. In yet further embodiments, an additional step of retrieving a sufficient quantity and quality of constructs, DNA or RNA after the permeabilization is involved.

Further these methods can employ detection protocols, including without limitation, PCR, Immuno-PCR and proximity ligation or proximity extension assay protocols, PEA, RCA, sequencing and fluorescence hybridization protocols.

In still further embodiments, the methods are high throughput methods. In some embodiments, the compositions described herein are used in high throughput protocols described herein. High throughput methods for detecting one or more targets in a sample (e.g., biological sample) can employ hundreds or thousands of wells containing the same or different samples. The method comprise contacting a sample (e.g., biological sample) with a composition comprising a first construct that comprises a first ligand that binds specifically to a first target, the first ligand attached or conjugated to a construct oligonucleotide sequence by a linker, where the construct oligonucleotide sequence comprises: an amplification handle, a barcode sequence that specifically identifies the first ligand from any other ligand that recognizes a different target, an optional unique molecular identifier sequence that is positioned adjacent to the barcode on its 5′ and/or 3′ end, and an anchor sequence (e.g., of at least 3 nucleotides) for hybridizing to a complementary sequence for generating a double stranded oligonucleotide sequence.

In similar embodiments, the composition comprises one or more substantially identical constructs, where each substantially identical first construct differs only in the sequence of the optional unique molecular identifier or its absence from a reference (e.g., “first” or “additional”) construct. In other embodiments, the composition comprises at least one additional construct, which comprises an additional ligand attached or conjugated to an additional construct oligonucleotide sequence by a linker. The additional ligand binds specifically to an additional target. The additional construct oligonucleotide sequence comprises: the same or different amplification handle, an additional barcode that specifically identifies the additional ligand; an optional additional unique molecular identifier that is positioned adjacent to the additional barcode on its 5′ or 3′ end, and the same or different anchor, where the additional target and the additional ligand, optional UMI, and additional barcode components differ from the corresponding components in any other construct in the composition.

High throughput protocols also involve washing the sample (e.g., biological sample) to remove unbound constructs; annealing the construct oligonucleotide sequence(s) through their respective anchors to the corresponding complementary sequences and generating double stranded oligonucleotide sequence(s). UMIs may also be optionally inserted to a position adjacent to the barcode on its 5′ and/or 3′ end before or after anchor hybridization.

Thereafter such methods involve detecting the construct barcode sequence(s) to identify whether the sample (e.g., biological sample; or samples present in individual wells) expresses or contains the first target, the additional targets, or a combination of first target and additional targets. Alternatively, expression level of the first target or additional targets in the sample occurs by detecting the amount of the corresponding barcodes.

In some embodiments, detection is performed by normalizing to the amount of a unique molecular identifier or the mean amount of two or more unique molecular identifiers.

The high throughput methods also can include adding the different compositions containing one or more first and additional constructs to the sample (e.g., biological sample) simultaneously or sequentially prior to the washing step. The methods can also include isolating the sample(s) (e.g., biological sample(s)) bound to one or more the first or additional constructs into individual cells or populations of cells after washing; and amplifying the double strand oligonucleotide sequence with primers annealed to amplification handles. Any of the other parameters of the compositions can be included that coordinate with the assay protocols used in the detection.

In yet other embodiments the use of compositions described here in detecting a target is discussed in the examples herein. The compositions described herein are designed and used to overcome the limitations of the currently existing methods for detecting and/or measuring RNA transcripts and proteins in single cells (i.e., droplet technology). The method referred to as Cellular Indexing of Transcriptome and Epitopes by sequencing (CITE-seq), disclosed in WO 2018/144813, uses compositions constructs comprising ligands attached or conjugated to polymer constructs, i.e., oligo-nucleotide sequences to simultaneously characterize the transcriptome and a potentially unlimited number of cell-surface markers from the same cell in a high-throughput manner. It combines unbiased genome-wide expression profiling with the measurement of specific protein markers in thousands of single cells using droplet microfluidics. The compositions can be used, in addition to adding an extra dimension to single-cell transcriptome data. This method provides a more detailed characterization of cell populations, but also allows study of post-transcriptional (and post-translational) gene regulation in single cells at an unprecedented depth.

Cellular processes and disease states can be understood with high information content single-cell transcriptomic, genomic, epigenomic, and proteomic profiling by performing the methods disclosed herein on mini-Drops in diverse laboratory settings. In some embodiments, the methods are useful to characterize the hematopoietic system. The methods disclosed herein allow in-depth characterization of single cells by simultaneous measurement of gene-expression levels and intracellular proteins, is highly scalable, only limited by the number of specific antibodies that are available and is compatible with other single-cell sequencing systems. Examples of single cell sequencing platforms suitable for integration with the compositions and methods described herein include the Drop-seq method, including, but not limited to, microfluidic, plate-based, or microwell, Seq-Well™ method and adaptations of the basic protocol, and InDrop™ method (1 Cell Bio). In other embodiments, a single cell sequencing platform suitable for integration with the methods and compositions described herein is 10× Genomics single cell 3′ solution (World Wide Web Uniform Resource Locator: 10×genomics.com/single-cell/), or single cell V(D)J solution (World Wide Web Uniform Resource Locator: 10×genomics.com/vdj/, either run on Chromium controller, or dedicated Chromium single cell controller). Still other useful sequencing protocols for combination with methods and compositions disclosed herein include Wafergen iCell8™ method (World Wide Web Uniform Resource Locator: wafergen.com/products/icell8-single-cell-system); Microwell-seq method, Fluidigm C1™ method and equivalent single cell products. Still other known sequencing protocols useful with the methods and compositions described herein include BD Resolve™ single cell analysis platform (derived from Cyto-seq) and ddSeq (from Illumina® Bio-Rad® SureCell™ WTA 3′ Library Prep Kit for the ddSEQ™ System, 2017, Pub. No. 1070-2016-014-B, Illumina Inc, Bio-Rad Laboratories, Inc.). In still other embodiments, the methods and compositions described herein are useful with combinatorial indexing based approaches (sci-RNA-seq™ method or SPLiT-seq™ method) and Spatial Transcriptomics, or comparable spatially resolved sequencing approaches. The compositions and methods described herein can also be used as an added layer of information on standard index sorting (FACS) and mRNA-sequencing-based approaches. In some embodiments, for example, standard FACS panels are supplemented with compositions disclosed herein that are detectable through plate-based sequencing. Still other sequencing protocols can be combined with the methods and compositions specifically described herein.

Thus, a high throughput method for characterizing a cell by simultaneous detection of one or more targets located in or on the cell and the transcriptome, genome, or epigenome involves contacting a sample (e.g., biological sample) containing cells with one or more of the composition as above described. In some embodiments, a composition that comprises a first ligand that binds specifically to a first target located in or on the surface of a cell, the first ligand is conjugated to an construct oligonucleotide sequence by a linker, where the construct oligonucleotide sequence comprises: a amplification handle; a barcode sequence that specifically identifies the first ligand from any other ligand that recognizes a different target, an optional unique molecular identifier sequence that is positioned adjacent to the barcode on its 5′ or 3′ end, and a polyA sequence of at least 3 nucleotides designed for hybridizing to a polyT sequence immobilized on a microfluidics bead (or a slide, a microwell, or a nanowell). In other embodiments, the composition comprises one or more substantially identical “first” constructs, where each substantially identical first construct differs only in the sequence of the optional unique molecular identifier or its absence from the reference “first” construct.

In still other embodiments, the composition further comprises at least one additional construct, which comprises an additional ligand conjugated to an additional construct oligonucleotide sequence by a linker, the additional ligand binding specifically to an additional target, and the additional construct oligonucleotide sequence comprising from 5′ to 3′: the amplification handle; an additional barcode that specifically identifies the additional ligand; an optional additional unique molecular identifier that is positioned adjacent to the additional barcode on its 5′ and/or 3′ end, and the anchor, where the additional components differ from the corresponding components in any other construct in the composition. The compositions can be added to the sample simultaneously or sequentially prior to a washing step. In other embodiments, the composition comprises one or more substantially identical “additional” constructs, where each substantially identical additional construct differs only in the sequence of the optional unique molecular identifier or its absence from the reference “additional” construct.

In such methods an individual single cell bound to one or more constructs is encapsulated into an aqueous droplet with one the bead, where each bead is conjugated to a construct comprising a unique cell barcode sequence comprising a 3′ polyT sequence. The single cell in each droplet is lysed, where mRNAs in the cell and the construct oligonucleotide sequence released from the ligand anneal to the polyT sequences of the bead. From the sequences annealed to the bead are generated double stranded cDNAs containing the cell barcode sequence and the reverse transcripts of the cellular mRNA and a double stranded DNA containing the cell barcode sequence and the construct oligonucleotide sequence(s). These methods can also include a step of optionally inserting one or more unique molecular identifiers to a position adjacent to the additional barcode on its 5′ and/or 3′ end before or after the annealing or hybridization step.

Further such methods involve creating by amplification a library containing the cDNA from the target cell's transcriptome, and the DNA containing the construct oligonucleotide sequence(s). In some embodiments, the construct barcode sequences are detected to identify whether the single cell expresses the first target (e.g., first epitope). In other embodiments of the method, the expression level of the first target (e.g., first epitope) in the single cell is determined by detecting the amount of the construct barcode. In yet other embodiments of the method, the detection is performed by normalization of the amount of any of the unique molecular identifiers or the mean amount of two or more unique molecular identifiers. Substantially simultaneously, the transcriptome of the library is associated with the cell identified by the binding and identification of the first and/or additional constructs.

Given the number of variations that one can generate in the constructs using the teachings provided herein, many other methods employing these compositions can be used for rapid and complex target identification.

To help define subsets of cells within a population of cells based on the cell's phenotype, it is essential to have an understanding of the presence or absence of specific intracellular protein markers and/or post-translational modifications of these markers. As demonstrated in the examples below, the methods and compositions described herein, provide in one aspect, a sequencing-based method that combines highly multiplexed ligand-based (e.g., antibody-based) detection of intracellular protein markers together with unbiased transcriptome profiling for thousands of single cells in parallel.

In further embodiments, as an additional step to any of the methods described herein, one may first perform a cell-hashtagging or batch barcoding step, by labelling every cell within a sample to be analyzed with the same “first construct” and then pooling multiply such hashtagged samples for further analysis. The further analysis includes analysis by any of the methods described herein. The oligonucleotide portions of the cell hashtag constructs, particularly the amplification handle sequences are different from those used in the “further” analytic methods, which permits cell hashtagging of samples subjected to those methods. This “hashtagging” method performed prior to pooling of samples subjected to additional analyses has several advantages. Multiplexing enables cost savings and the ability to control for batch effects—for example, process treated/untreated at the same time. The cell hashtag constructs allow unequivocal determination of most doublets. Finally, the combination of these two advantages enables the overload of droplet-based scRNAseq experiments (i.e, use 20,000 cells, rather than 4,000 cells, per lane), resulting in decreased cost of experiments and increased information produced by the experiments. This hashtagging embodiment can be used to multiplex samples of the same genotype without the need to perform genotyping on samples.

The methods and compositions described herein can be used for spatial, bulk, and multiplexed detection of nucleic acid and protein targets in a sample (e.g., biological sample), by employing the assays and methods disclosed herein.

### Certain Terms Used Herein

Unless otherwise defined, all technical and scientific terms used herein have the same meaning as commonly understood by one of ordinary skill in the art. Preferred methods and compositions are described herein, although methods and compositions similar or equivalent to those described herein can be used in practice or testing of the present technology. All publications, patent applications, patents and other references mentioned herein are incorporated by reference in their entirety. The methods, compositions, and examples disclosed herein are illustrative only and not intended to be limiting.

The term “construct” generally refers to a chemically synthesized or genetically engineered assemblage that comprises a ligand attached (covalently, non-covalently, or otherwise as noted herein) to at least one polymer construct (e.g., in some embodiments, an oligonucleotide sequence) by a linker. Each polymer construct comprises several functional elements: an amplification handle; a barcode that specifically identifies the attached ligand, an optional unique molecular identifier that is positioned adjacent to the barcode on its 5′ or 3′ end, and an anchor for hybridizing to a capture sequence that comprises a sequence complementary to the anchor. These components of the construct can occur in any order. In some embodiments, the components are listed 5′ to 3′: ligand, linker, amplification handle, barcode, and anchor with the UMI on either end of the barcode. In other embodiments, the components are listed 3′ to 5′: ligand, linker, amplification handle, barcode, and anchor with the UMI on either end of the barcode. In still other embodiments, these elements of the construct can be in any other order. In still other embodiments, a construct comprises a single ligand linked to multiple identical polymer constructs. In some embodiments, each polymer construct is directly linked to the ligand (one linkage per polymer construct). In some embodiments, the polymer constructs are linked to the ligand as concatamers (multiple polymer constructs per single ligand linkage). For example, a single ligand (i.e., a monoclonal antibody) may be linked to from 1 to 50 polymer constructs.

The term “polymer” generally refers to any backbone of multiple monomeric components that can function to bind to the selected ligand and/or anchor component and be utilized in a downstream assay. This assay may utilize the activity of one or more enzymes, for example reverse transcriptases, DNA or RNA polymerases, DNA or RNA ligases, etc. Such polymers or monomeric components include oligonucleotides (e.g., DNA, RNA, synthetic or recombinant DNA or RNA bases or analogs of DNA or RNA bases), peptide nucleic acids (i.e., a synthetic nucleic acid analog in which natural nucleotide bases are linked to a peptide-like backbone instead of the sugar-phosphate backbone found in DNA and RNA), locked nucleic acids (LNA; see, e.g., Grunweller A and Hartmann R K, “Locked nucleic acid oligonucleotides: the next generation of antisense agents?”, BioDrugs 2007. 21(4):235-43)), or polyamide polymers (see, e.g. Dervan P B and Burli, R W, Sequence-specific DNA recognition by polyamides”, Curr. Opn Chem. Biol. 1999, 3:688-693). For simplicity and ease of understanding, throughout this specification a polymer construct or a functional component thereof (e.g., anchor, barcode, UMI or amplification handle) may also be exemplified as a specific polymer or monomeric component, such as an oligonucleotide sequence, a nucleic acid, a nucleic acid sequence, etc. However, wherever the term “oligonucleotide”, “nucleic acid” or nucleotide” or a similar specific example of a monomer or polymer is used in this specification, it should also be understood to mean that the polymer construct or component may be formed of any suitable polymer as described in this paragraph.

The terms “first”, “additional” and “substantially identical” are used throughout this specification generally as reference terms to distinguish between various forms and components of constructs. “First construct” generally refers to a construct with these specified components in which a single specified “first” ligand binds a specific “first” target. The “first” barcode is specific for the first ligand; the UMI identifies only that “first” polymer construct, and the anchor binds a specified complementary sequence. The term “additional construct” generally refers to a construct that differs from any other construct used in the compositions and methods defined herein in the identity of the target, ligand, and barcode. In some embodiments, an additional construct differs from other constructs in the compositions or methods by the identity of target, ligand, barcode, UMI and anchor. Each additional construct comprises an additional ligand attached or conjugated to an additional polymer construct by a linker. The additional ligand binds specifically to an additional target different from that of the first target. The linker between the ligand and the additional polymer construct may be the same or different from the linker in the first construct. The additional polymer construct also differs in the identity of its functional elements. The amplification handle may be the same or different from that used in the first construct. However, the additional barcode that specifically identifies the additional ligand, does not identify any other ligand. The optional additional UMI that is positioned adjacent to the additional barcode on its 5′ or 3′ end, is specific for the additional polymer construct. In yet other embodiments, the additional anchor has the same or a different sequence for hybridizing to the same or a different capture complementary sequence than that to which the first anchor binds. In some embodiments, each “additional” ligand, “additional” target, “additional” barcode and “additional” UMI components of each additional construct differs from the corresponding component in any other construct in the described method or composition.

The term “substantially identical” construct generally refers to a number of constructs or components, which differ from a reference construct, e.g., the “first” construct or a specific additional construct, only in the sequence of the optional unique molecular identifier or its absence from the construct. In some embodiments, each one of the substantially identical constructs shares the same target, ligand, amplification handle, barcode and anchor as does the reference (first or additional) construct. In some embodiments, each one of the substantially identical constructs shares the same target, ligand, barcode and anchor as does the reference (first or additional) construct. In some embodiments a substantially identical “first” construct differs from the reference “first” construct in the sequence and/or presence of the UMI. In other embodiments, the substantially identical additional construct differs from the reference additional construct in the UMI and the amplification handle.

The term “attachment” or “attach” generally describes the interaction between the components of the constructs is meant covalent attachments or a variety of non-covalent types of attachment. Other attachment chemistries useful in assembling the constructs described herein include, but are not limited to, thiol-maleimide, thiol-haloacetate, amine-NHS, amine-isothiocyanate, azide-alkyne (CuAAC), tetrazole-cyclooctene (iEDDA).

The term “target” generally refers to any naturally occurring or synthetic biological or chemical molecule, in some embodiments, the target refers to any biological or chemical molecule expressed on the surface of a cell. In some embodiments, the target refers to any biological or chemical molecule expressed on the surface of a cell. In other embodiments, the target refers to any biological or chemical molecule expressed intracellularly. In some embodiments, the target refers to any biological or chemical molecule occurring in a naturally occurring, synthetic, recombinantly engineered or isolated library, panel, or mixture of targets. In some embodiments, the target refers to any biological or chemical molecule occurring in a sample (e.g., biological sample). The corresponding terms “first target” and each “additional target” generally refer to different targets. The first and additional targets, may independently be selected from a peptide, a protein, an antibody or antibody fragment, an affibody, a ribo- or deoxyribo-nucleic acid sequence, an aptamer, a lipid, a polysaccharide, a lectin, or a chimeric molecule formed of multiples of the same or different targets. In the examples below, the targets are intracellular antigens or epitopes.

The term “sample” or “biological sample” generally refers to a naturally-occurring sample or deliberately designed or synthesized sample or library containing the selected target. In some embodiments, the sample contains a population of cells or cell fragments, including without limitation cell membrane components, exosomes, and sub-cellular components. The cells may be a homogenous population of cells, such as isolated cells of a particular type, or a mixture of different cell types, such as from a biological fluid or tissue of a human or mammalian or other species subject. Still other samples for use in the methods and with the compositions include, without limitation, blood samples, including serum, plasma, whole blood, and peripheral blood, saliva, urine, vaginal or cervical secretions, amniotic fluid, placental fluid, cerebrospinal fluid, or serous fluids, mucosal secretions (e.g., buccal, vaginal or rectal). Still other samples include a blood-derived or biopsy-derived biological sample of tissue or a cell lysate (i.e., a mixture derived from tissue and/or cells). Other suitable tissue includes hair, fingernails and the like. Still other samples include libraries of antibodies, antibody fragments and antibody mimetics like affibodies. Such samples may further be diluted with saline, buffer or a physiologically acceptable diluent. Alternatively, such samples are concentrated by conventional means. Still other samples can be synthesized or engineered collections of chemical molecules, proteins, antibodies or any other of the targets described herein.

The term “ligand” generally refers to any naturally occurring or synthetic biological or chemical molecule which is used to bind specifically to a single identified target. The binding can be covalently or non-covalent, i.e., conjugated or by any known means taking into account the nature of the ligand and its respective target. The terms “first ligand” and “additional ligand” refer to ligands that bind to different targets or different portions of a target. For example, multiple “first ligands” bind to the same target at the same site. Multiple additional ligands bind to a target different than the first ligand and different than any additional ligand. The first and additional ligands, may be independently selected from a peptide, a protein, an antibody or antibody fragment, an antibody mimetic, an affibody, a ribo- or deoxyribo-nucleic acid sequence, an aptamer, a lipid, a polysaccharide, a lectin, or a chimeric molecule formed of multiples of the same or different the first ligands. In some embodiments, the ligand(s) of the constructs can also be directly labeled with one or more detectable labels, such as fluorophores (see labels discussed below) that can be measured by methods independent of the methods of measuring or detecting the polymer construct described otherwise herein.

The term “detectable label” generally refers to a reagent, moiety or compound capable of providing a detectable signal, depending upon the assay format employed. A label may be associated with the construct as a whole, or with the ligand only, or with the polymer construct or a functional portion thereof. Alternatively, different labels may be used for each component of the construct. Such labels are capable, alone or in concert with other compositions or compounds, of providing a detectable signal. In some embodiments, the labels are desirably interactive to produce a detectable signal. Most desirably, the label is detectable visually, e.g. colorimetrically. A variety of enzyme systems operate to reveal a colorimetric signal in an assay, e.g., glucose oxidase (which uses glucose as a substrate) releases peroxide as a product that in the presence of peroxidase and a hydrogen donor such as tetramethyl benzidine (TMB) produces an oxidized TMB that is seen as a blue color. Other examples include horseradish peroxidase (HRP) or alkaline phosphatase (AP), and hexokinase in conjunction with glucose-6-phosphate dehydrogenase that reacts with ATP, glucose, and NAD+ to yield, among other products, NADH that is detected as increased absorbance at 340 nm wavelength. Still other label systems that may be utilized in the described methods and constructs are detectable by other means, e.g., colored latex microparticles (Bangs Laboratories, Indiana) in which a dye is embedded may be used in place of enzymes to provide a visual signal indicative of the presence of the labeled ligand or construct in applicable assays. Still other labels include fluorescent compounds, fluorophores, radioactive compounds or elements. In some embodiments, a fluorescent detectable fluorochrome, e.g., fluorescein isothiocyanate (FITC), phycoerythrin (PE), allophycocyanin (APC), coriphosphine-O (CPO) or tandem dyes, PE-cyanin-5 or -7 (PC5 or PC7)), PE-Texas Red (ECD), PE-cyanin-5.5, rhodamine, PerCP, and Alexa dyes. Combinations of such labels, such as Texas Red and rhodamine, FITC+PE, FITC+PECy5 and PE+PECy7, among others may be used depending upon assay method. The selection and/or generation of suitable labels for use in labeling the ligand and/or any component of the polymer construct is within the skill of the art, provided with this specification.

Other components of the methods and compositions described herein can also be detectably labeled. Additionally, or alternatively to the labeling of the ligand, the polymer construct(s) can be labeled with one or more detectable labels, such as fluorophores and other labels defined below. The detection of these labels is performed by methods independent of the methods described herein for measurement of the polymer construct or its components. Additionally, or alternatively, the ligand and polymer construct(s) can be labeled so that when assembled into the final construct, the successful assembly is detectable, such as for production of the final construct. Additionally, or alternatively, in the methods described below, the capture polymer can be labeled with one or more detectable labels. Additionally, or alternatively, detectable labels can be used in the methods described below, to provide indications of successful binding. For example, the substrate to which the capture polymer is immobilized can be labeled with one or more detectable labels. Additionally, or alternatively, one or more detectable labels can be used to show successful binding of the capture polymer and the polymer construct. In some embodiments, the successful binding of the capture polymer to the substrate can be labeled. Additionally, or alternatively, the successful association of the polymer construct and the substrate to which the capture polymer is immobilized can be labeled with one or more detectable labels. Still further, such labels can be used to indicate the successful association of the ligand and the capture polymer. Additionally, or alternatively, such labels can be used to indicate the association of the ligand and the substrate to which the capture polymer is immobilized. Still other uses of the detectable labels in these methods and compositions are contemplated.

The term “antibody or fragment” generally refers to a monoclonal antibody, a synthetic antibody, a recombinant antibody, a chimeric antibody, a humanized antibody, a human antibody, a CDR-grafted antibody, a multispecific binding construct that can bind two or more targets, a dual specific antibody, a bi-specific antibody or a multi-specific antibody, or an affinity matured antibody, a single antibody chain or an scFv fragment, a diabody, a single chain comprising complementary scFvs (tandem scFvs) or bispecific tandem scFvs, an Fv construct, a disulfide-linked Fv, a Fab construct, a Fab′ construct, a F(ab′)2 construct, an Fc construct, a monovalent or bivalent construct from which domains non-essential to monoclonal antibody function have been removed, a single-chain molecule containing one V_{L}, one V_{H} antigen-binding domain, and one or two constant “effector” domains optionally connected by linker domains, a univalent antibody lacking a hinge region, a single domain antibody, a dual variable domain immunoglobulin (DVD-Ig) binding protein or a nanobody. Also included in this definition are antibody mimetics such as affibodies, i.e., a class of engineered affinity proteins, generally small (˜6.5 kDa) single domain proteins that can be isolated for high affinity and specificity to any given protein target.

The term “linker” generally refers to any moiety used to attach or associate the ligand to the polymer construct/oligonucleotide sequence portion of the constructs. Thus in some embodiments, the linker is a covalent bond. In other embodiments, the linker is a non-covalent bond. In some embodiments, the linker is composed of at least one to about 25 atoms. Thus in various embodiments, the linker is formed of at least 2, 3, 4, 5, 6, 7, 8, 9, 10, 11, 12, 13, 14, 15, 16, 17, 18, 19, 20, 21, 22, 23, 24 or 25 atoms. In still other embodiments, the linker is at least one to about 60 nucleic acids. Thus in various embodiments, the linker is formed of a sequence of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, up to 60 nucleic acids. In yet other embodiments, the linker refers to at least one to about 30 amino acids. Thus in various embodiments, the linker is formed of a sequence of at least 2, 3, 4, 5, 6, 7, 8, 9, 10, 11, 12, 13, 14, 15, 16, 17, 18, 19, 20, 21, 22, 23, 24, 25, 26, 27, 28, 29, up to about 30 amino acids. In still other embodiments, the linker can be a larger compound or two or more compounds that associate covalently or non-covalently. In still other embodiments, the linker can be a combination of the linkers defined herein. The linkers used in the constructs of the compositions and methods are in some embodiments cleavable. The linkers used in the constructs of the compositions and methods are in some embodiments non-cleavable. Without limitation, in some embodiments, the linker is a disulfide bond. In some examples, the linker comprises a complex of biotin bound to the construct oligonucleotide sequence by a disulfide bond, with streptavidin fused to the ligand. In some embodiments, the biotin is bound to the ligand and the streptavidin is fused to the construct oligonucleotide sequence. Although examples show the linker bound to the 5′ end of the oligonucleotide of the construct, in some embodiments, the linker may be covalently attached or conjugated other than covalently to any oligonucleotide sequence portion of the construct. In yet other embodiments, when the ligand is a recombinant or synthesized antibody, the linker can be engineered into the antibody sequence to facilitate 1:1 coupling to the polymer construct, thereby simplifying manufacturing of the ligand, the construct and/or the polymer construct. For example, a Halotag® linker can be engineered into the selected ligand (e.g., antibody) or into the polymer construct or component, for such purposes. Additionally, or alternatively, the ligand is linked to the polymer construct upon production in the same cell. See, e.g., the Halotag® protocols described by Flexi® Vector Systems Technical Manual (TM254-revised 5/17), copyright 2017 by Promega Corporation; and Janssen D. B., Evolving haloalkaline dehalogenase, Curr. Opin. Chem. Biol., 2004, 8:150-159.

The term “polymer construct” or “construct oligonucleotide sequence” generally refers to the portion of the construct which is associated with the ligand. As stated above, this association can be covalent, non-covalent or by any suitable conjugation and employing any suitable linker. The polymer construct is formed by a series of functional polymeric elements, e.g., nucleic acid sequences or other polymers as defined above, each having a function as defined herein. The ligand can be attached to the construct oligonucleotide sequence at its 5′ end or at any other portion, provided that the attachment or conjugation does not prevent the functions of the components of the construct oligonucleotide sequence. These components are for each “first” or “additional” construct oligonucleotide sequence, an amplification handle; a barcode, an optional UMI and an anchor. In general, the polymer construct can be any length that accommodates the lengths of its functional components. In some embodiments, the polymer construct is between 20 and 100 monomeric components, e.g., nucleic acid bases, in length. In some embodiments, the construct oligonucleotide sequence is at least 20, 30, 40, 50, 60, 70, 80, 90 or over 100 monomeric components, e.g., nucleic acid bases, in length. In other embodiments, the construct oligonucleotide is 200 to about 400 monomeric components, e.g., nucleotides, in length. In some embodiments, the polymer construct is generally made up of deoxyribonucleic acids (DNA). In some embodiments, the construct oligonucleotide is a DNA sequence. In other embodiments, the construct oligonucleotide, or portions thereof, comprises modified DNA bases. Modification of DNA bases are known in the art, and can include chemically modified bases including labels. In some embodiments, the construct oligonucleotide, or portions thereof, comprises ribonucleic acid (RNA) sequences or modified ribonucleotide bases. Modification of RNA bases are known in the art, and can include chemically modified bases including labels. In still other embodiments, different portions of the construct oligonucleotide sequence can comprise DNA and RNA, modified bases, or modified polymer connections (including but not limited to PNAs and LNAs). For a description of modifications to oligonucleotides, see commercial suppliers, e.g., Integrated DNA Technologies, USA website; Custom Oligonucleotide Modifications Guide, Sigma-Aldrich, World Wide Web Uniform Resource Locator: sigmaaldrich.com/technical-documents/articles/biology/custom-dna-oligos-modifications.html, and Modified Oligonucleotides, TriLink, World Wide Web Uniform Resource Locator: trilinkbiotech.com/oligo/modifiedoligos.asp. As described above, in still other embodiments, the polymer construct is composed of polyamides, PNA, etc.

The term “amplification handle” generally refers to a functional component of the construct oligonucleotide sequence which itself is an oligonucleotide or polynucleotide sequence that provides an annealing site for amplification of the construct oligonucleotide sequence. The amplification handle can be formed of polymers of DNA, RNA, PNA, modified bases or combinations of these bases, or polyamides, etc. In some embodiments, the amplification handle is about 10 of such monomeric components, e.g., nucleotide bases, in length. In other embodiments, the amplification handle is at least about 5 to 100 monomeric components, e.g., nucleotides, in length. Thus in various embodiments, the amplification handle is formed of a sequence of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In some embodiments, when present in first or additional construct oligonucleotide sequences, the amplification handle can be the same or different, depending upon the techniques intended to be used for amplification. In certain embodiments, the amplification handle can be a generic sequence suitable as a annealing site for a variety of amplification technologies. Amplification technologies include, but are not limited to, DNA-polymerase based amplification systems, such as polymerase chain reaction (PCR), real-time PCR, loop mediated isothermal amplification (LAMP, MALBAC), strand displacement amplification (SDA), multiple displacement amplification (MDA), recombinase polymerase amplification (RPA) and polymerization by any number of DNA polymerases (for example, T4 DNA polymerase, Sulfulobus DNA polymerase, Klenow DNA polymerase, Bst polymerase, Phi29 polymerase) and RNA-polymerase based amplification systems (such as T7-, T3-, and SP6-RNA-polymerase amplification), nucleic acid sequence based amplification (NASBA), self-sustained sequence replication (3SR), rolling circle amplification (RCA), ligase chain reaction (LCR), helicase dependent amplification (HDA), ramification amplification method and RNA-seq.

The term “barcode” or “construct barcode” generally describes a defined polymer, e.g., a polynucleotide, which when it is a functional element of the polymer construct, is specific for a single ligand. As used in the various methods described herein the term barcode can be a “cell barcode” or “substrate barcode”, which describes a defined polynucleotide, specific for identifying a particular cell or substrate, e.g., Drop-seq microbead. In some embodiments, the barcode can be formed of a defined sequence of DNA, RNA, modified bases or combinations of these bases, as well as any other polymer defined above. In some embodiments, the barcode is about 2 to 4 monomeric components, e.g., nucleotide bases, in length. In other embodiments, the barcode is at least about 1 to 100 monomeric components, e.g., nucleotides, in length. Thus in various embodiments, the barcode is formed of a sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

The term “unique molecular identifier” (UMI), also called equivalently a “Random Molecular Tag” (RMT), generally refers to a random sequence of monomeric components of a polymer as described above, e.g., nucleotide bases, which when it is a functional element of the polymer construct, is specific for that polymer construct. The UMI permits identification of amplification duplicates of the polymer construct/construct oligonucleotide sequence with which it is associated. In the description of the methods and compositions herein, one or more UMI may be associated with a single polymer construct/construct oligonucleotide sequence. The UMI may be positioned 5′ or 3′ to the barcode in the composition. In some embodiments, the UMI may be inserted into the polymer/construct oligonucleotide sequence as part of the described methods. In some embodiments of the methods described herein, depending on which RNA-sequencing method is used, a UMI is added during the method. However, not all RNA-seq methods make use of UMIs. In some examples of single cell droplet RNA-sequencing, another UMI is introduced during reverse transcription. Each UMI is specific for its construct oligonucleotide sequence. Thus when the compositions or methods comprise multiple “first constructs”, each first construct differs only in the sequence of its UMI. Each additional construct will also have its own UMI, which is not present on duplicate additional constructs or additional constructs that differ from each other in target, ligand, barcode and anchor specificity. Similarly, as used in the various methods described herein, a UMI may be associated with a polymer, e.g., an oligo or polynucleotide sequence, used in a particular assay format or with a polymer, e.g., an oligo or polynucleotide, that is immobilized on a substrate. Each UMI for each polymer construct, e.g., oligonucleotide or polynucleotide, is different from any other UMI used in the compositions or methods. In some embodiments, the UMI is formed of a random sequence of DNA, RNA, modified bases or combinations of these bases or other monomers of the polymers identified above. In some embodiments, a UMI is about 8 monomeric components, e.g., nucleotides, in length. In other embodiments, each UMI can be at least about 1 to 100 monomeric components, e.g., nucleotides, in length. Thus in various embodiments, the UMI is formed of a random sequence of at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids.

The term “anchor” generally refers to a defined polymer, e.g., a polynucleotide or oligonucleotide sequence, which is designed to hybridize to a capture polymer, e.g., oligonucleotide sequence. In some embodiments of the polymer construct, the anchor is designed for the purpose of generating a double stranded construct oligonucleotide sequence. In some embodiments, the anchor is positioned at the 3′ end of the construct oligonucleotide sequence. In other embodiments, the anchor is positioned at the 5′ end of the construct oligonucleotide sequence. Each anchor is specific for its intended complementary sequence. When the compositions or methods comprise multiple “first constructs”, each first construct has the same anchor sequence. In some embodiments, each additional anchor has a different additional sequence which hybridizes to a different complementary sequence. In other embodiments, each additional anchor may have the same anchor sequence as the first or other constructs, depending upon the assay method steps. When used in the various methods described herein, an anchor may hybridize to a free complementary sequence or with a complementary sequence that is immobilized on a substrate. In certain embodiments, the anchor can be formed of a sequence of monomers of the selected polymer, e.g., DNA, RNA, modified bases or combinations of these bases, PNAs, polyamides, etc. In some embodiments, an anchor is about 3 to 15 monomeric components, e.g., nucleotides, in length. In other embodiments, each anchor can be at least about 3 to 100 monomeric components, e.g., nucleotides, in length. Thus in various embodiments, the anchor is formed of a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric components, e.g., nucleic acids. In some embodiments, and as shown in the examples, an anchor sequence is a polyA sequence. In other embodiments, an anchor sequence is a polyT sequence. In still other embodiments, the anchor sequence may be a random sequence provided that it can hybridize to its intended complementary sequence.

The term “capture oligonucleotide” or “capture oligo” or “capture polymer” generally refers a polymeric sequence, e.g., an oligonucleotide, containing a sequence that is complementary to the anchor. The capture polymer/oligo is not part of the first or additional constructs; rather it is any polymeric sequence or oligonucleotide belonging to a construct-purification kit or an mRNA-sequencing kit. As used herein, the term “complementary sequence” refers to the sequence to which the anchor sequence is intended to hybridize to generate amplification and the generation of double stranded sequencing. In certain embodiments, the capture polymer/oligonucleotide sequence may contain sequences that can be used as amplification handles and optionally one or more unique molecular identifiers and barcode sequences. In the methods described herein, the extension of the capture polymer/oligonucleotide with its complementary sequence hybridized to the anchor sequence copies the barcode, the UMI and the amplification handle from the first or additional constructs onto the capture polymer/oligonucleotide. In some embodiments, the capture polymer/oligonucleotide and its complementary sequence can be formed of DNA, RNA, modified bases or combinations of these bases or of any other polymeric component as defined above. Depending upon the assay steps involved and the intended target, the capture sequence can be unhindered or “free” in the sample. In some embodiments, the capture polymer/oligo contains a complementary sequence that is a primer sequence designed to participate in amplifying the polymer construct/construct oligonucleotide sequence. In other embodiments, the capture sequence is immobilized on a substrate. Similarly, to the anchor sequence, each capture sequence can be at least about 3 to about 100 monomeric units, e.g., nucleotides, in length. Thus in various embodiments, the capture or its complementary sequence is formed of a sequence of at least 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 80, 91, 92, 93, 94, 95, 96, 97, 98, 99 or up to 100 monomeric units, e.g., nucleic acids. In some embodiments, and as shown in the examples, a capture oligo contains a complementary sequence polyT sequence when the anchor sequence is a polyA sequence. In other embodiments, the capture oligo contains a polyA sequence. In still other embodiments, the complementary sequence may be a random polymer, e.g., oligonucleotide sequence, provided that it can hybridize to its intended anchor sequence.

The term “cell hashtagging”, “cell hashing” or “batch barcoding” generally refers to using one first construct as described above to label all cells in a sample prior to pooling multiple samples of cells and prior to performing other scRNA seq or CITE-seq methods using other such constructs having different amplification handle sequences. Upon reverse-transcription, the oligonucleotide portions of the cell hashtag constructs are converted to “hashtags” which enable identification and assignment of each cell within a heterogeneous mixture to its respective original population. The cell-hashtag construct thus serves the purposes of identifying all the cells of a particular sample. The ligand in the cell-hashtag construct can be a pool of antibodies to broadly expressed proteins or a single antibody to such a protein, or any other cell-binding ligand. Because the amplification handle sequence of the cell hashtag is different from that of the first or additional construct used in the CITE-Seq methods, one may follow individual cells of an identified sample through the CITE-Seq methods, which are typically used to identify cells within a sample that differentially express specific cell surface proteins.

The term “immobilized” generally means that the capture polymer/oligonucleotide sequence is attached to a solid substrate resulting in reduction or loss of mobility via physical adsorption through charge-charge interaction or hydrophobic interaction, covalent bonding, Streptavidin-Biotin interaction or affinity coupling.

The term “substrate” generally means a microparticle (bead), a microfluidics microparticle (bead), a slide, a multi-well plate, a microwell, a nanowell, or a chip. The substrates are conventional and can be glass, plastic or of any conventional materials suitable for the particular assay or diagnostic protocols.

The term “phosphorothioate (PS) bond” substitutes a sulfur atom for a non-bridging oxygen in the phosphate backbone of an oligo. This modification renders the internucleotide linkage resistant to nuclease degradation. A “phosphorothioate (PS) bond” is indicated with a * in Example 1 and refers to a phosphorothioate (PS) bond between the two nucleotide residues.

For simplicity and ease of understanding, throughout this specification, certain specific examples are provided to teach the construction, use and operation of the various elements of the compositions and methods described herein. Such specific examples are not intended to limit the scope of this description.

### EXAMPLES

The examples set forth below illustrate certain embodiments and do not limit the technology.

### Example 1: Antibody Constructs

Nucleic acid sequences for antibody-derived tags (ADTs) described herein are provided in the tables below. ADTs in the “A set” are useful for applications that use dT capture, and ADTs in the “B set” are useful for 10× 3′ single cell assays.

| | | | SEQ |
|---|---|---|---|
| | | | ID |
| ADT | antigen | nucleotide sequence | NO |
| A1 | HU.CD19 | CCTTGGCACCCGAGAATTCCACTGGGCAATTACTCGBAAAAAAAAAAA | 1 |
| | | AAAAAAAAAAAAAAAAAAA*A*A | |
| A2 | Hu.CD3 | CCTTGGCACCCGAGAATTCCACTCATTGTAACTCCTBAAAAAAAAAAAA | 2 |
| | | AAAAAAAAAAAAAAAAAA*A*A | |
| A3 | HU.CD16 | CCTTGGCACCCGAGAATTCCAAAGTTCACTCTTTGCBAAAAAAAAAAAA | 3 |
| | | AAAAAAAAAAAAAAAAAA*A*A | |
| A4 | Hu.CD4 | CCTTGGCACCCGAGAATTCCATGTTCCCGCTCAACTBAAAAAAAAAAA | 4 |
| | | AAAAAAAAAAAAAAAAAAA*A*A | |
| A5 | Hu.CD11c | CCTTGGCACCCGAGAATTCCATACGCCTATAACTTGBAAAAAAAAAAA | 5 |
| | | AAAAAAAAAAAAAAAAAAA*A*A | |
| A6 | HU.CD56 | CCTTGGCACCCGAGAATTCCATCCTTTCCTGATAGGBAAAAAAAAAAA | 6 |
| | | AAAAAAAAAAAAAAAAAAA*A*A | |
| A7 | HU.CD14 | CCTTGGCACCCGAGAATTCCATCTCAGACCTCCGTABAAAAAAAAAAA | 7 |
| | | AAAAAAAAAAAAAAAAAAA*A*A | |
| A8 | Hu.CD8 | CCTTGGCACCCGAGAATTCCAGCGCAACTTGATGATBAAAAAAAAAAA | 8 |
| | | AAAAAAAAAAAAAAAAAAA*A*A | |
| A9 | Hu.CD45 | CCTTGGCACCCGAGAATTCCATCCCTTGCGATTTACBAAAAAAAAAAA | 9 |
| | | AAAAAAAAAAAAAAAAAAA*A*A | |
| A10 | Hu.ZAP70 | CCTTGGCACCCGAGAATTCCACTTGCCTGCAATCACBAAAAAAAAAAA | 10 |
| | | AAAAAAAAAAAAAAAAAAA*A*A | |
| A11 | Hu.Perforin | CCTTGGCACCCGAGAATTCCATCTTCGTTTGAACGGBAAAAAAAAAAA | 11 |
| | | AAAAAAAAAAAAAAAAAAA*A*A | |
| A12 | Hu.GrzmB | CCTTGGCACCCGAGAATTCCAGCGTGTTGTGGTATTBAAAAAAAAAAA | 12 |
| | | AAAAAAAAAAAAAAAAAAA*A*A | |
| B1 | Hu.CD19 | GTGACTGGAGTTCAGACGTGTGCTCTTCCGATCTNNNNNNNNNNCTG | 13 |
| | | GGCAATTACTCGNNNNNNNNNGCTTTAAGGCCGGTCCTAGC*A*A | |
| B2 | Hu.CD3 | GTGACTGGAGTTCAGACGTGTGCTCTTCCGATCTNNNNNNNNNNCTC | 14 |
| | | ATTGTAACTCCTNNNNNNNNNGCTTTAAGGCCGGTCCTAGC*A*A | |
| B3 | Hu.CD16 | GTGACTGGAGTTCAGACGTGTGCTCTTCCGATCTNNNNNNNNNNAAG | 15 |
| | | TTCACTCTTTGCNNNNNNNNNGCTTTAAGGCCGGTCCTAGC*A*A | |
| B4 | Hu.CD4 | GTGACTGGAGTTCAGACGTGTGCTCTTCCGATCTNNNNNNNNNNTGTT | 16 |
| | | CCCGCTCAACTNNNNNNNNNGCTTTAAGGCCGGTCCTAGC*A*A | |
| B5 | Hu.CD11c | GTGACTGGAGTTCAGACGTGTGCTCTTCCGATCTNNNNNNNNNNTAC | 17 |
| | | GCCTATAACTTGNNNNNNNNNGCTTTAAGGCCGGTCCTAGC*A*A | |
| B6 | HU.CD56 | GTGACTGGAGTTCAGACGTGTGCTCTTCCGATCTNNNNNNNNNNTCCT | 18 |
| | | TTCCTGATAGGNNNNNNNNNGCTTTAAGGCCGGTCCTAGC*A*A | |
| B7 | HU.CD14 | GTGACTGGAGTTCAGACGTGTGCTCTTCCGATCTNNNNNNNNNNTCTC | 19 |
| | | AGACCTCCGTANNNNNNNNNGCTTTAAGGCCGGTCCTAGC*A*A | |
| B8 | Hu.CD8 | GTGACTGGAGTTCAGACGTGTGCTCTTCCGATCTNNNNNNNNNNGCG | 20 |
| | | CAACTTGATGATNNNNNNNNNGCTTTAAGGCCGGTCCTAGC*A*A | |
| B9 | Hu.CD45 | GTGACTGGAGTTCAGACGTGTGCTCTTCCGATCTNNNNNNNNNNTCC | 21 |
| | | CTTGCGATTTACNNNNNNNNNGCTTTAAGGCCGGTCCTAGC*A*A | |
| B10 | Hu.ZAP70 | GTGACTGGAGTTCAGACGTGTGCTCTTCCGATCTNNNNNNNNNNCTT | 22 |
| | | GCCTGCAATCACNNNNNNNNNGCTTTAAGGCCGGTCCTAGC*A*A | |
| B11 | Hu.Perforin | GTGACTGGAGTTCAGACGTGTGCTCTTCCGATCTNNNNNNNNNNTCTT | 23 |
| | | CGTTTGAACGGNNNNNNNNNGCTTTAAGGCCGGTCCTAGC*A*A | |
| B12 | Hu.GrzmB | GTGACTGGAGTTCAGACGTGTGCTCTTCCGATCTNNNNNNNNNNGCG | 24 |
| | | TGTTGTGGTATTNNNNNNNNNGCTTTAAGGCCGGTCCTAGC*A*A | |

| | | | | SEQ ID |
|---|---|---|---|---|
| ADT | antigen | component | nucleotide sequence | NO |
| B1 | Hu.CD19 | 5’ | GTGACTGGAGTTCAGACGTGTGCTCTTCCGATCTNNNN | 25 |
| | | | NNNNNN | |
| | | barcode | CTGGGCAATTACTCG | 26 |
| | | 3’ | NNNNNNNNNGCTTTAAGGCCGGTCCTAGC*A*A | 27 |
| B2 | Hu.CD3 | 5’ | GTGACTGGAGTTCAGACGTGTGCTCTTCCGATCTNNNN | 28 |
| | | | NNNNNN | |
| | | barcode | CTCATTGTAACTCCT | 29 |
| | | 3’ | NNNNNNNNNGCTTTAAGGCCGGTCCTAGC*A*A | 30 |
| B3 | Hu.CD16 | 5’ | GTGACTGGAGTTCAGACGTGTGCTCTTCCGATCTNNNN | 31 |
| | | | NNNNNN | |
| | | barcode | AAGTTCACTCTTTGC | 32 |
| | | 3’ | NNNNNNNNNGCTTTAAGGCCGGTCCTAGC*A*A | 33 |
| B4 | Hu.CD4 | 5’ | GTGACTGGAGTTCAGACGTGTGCTCTTCCGATCTNNNN | 34 |
| | | | NNNNNN | |
| | | barcode | TGTTCCCGCTCAACT | 35 |
| | | 3’ | NNNNNNNNNGCTTTAAGGCCGGTCCTAGC*A*A | 36 |
| B5 | Hu.CD11c | 5’ | GTGACTGGAGTTCAGACGTGTGCTCTTCCGATCTNNNN | 37 |
| | | | NNNNNN | |
| | | barcode | TACGCCTATAACTTG | 38 |
| | | 3’ | NNNNNNNNNGCTTTAAGGCCGGTCCTAGC*A*A | 39 |
| B6 | HU.CD56 | 5’ | GTGACTGGAGTTCAGACGTGTGCTCTTCCGATCTNNNN | 40 |
| | | | NNNNNN | |
| | | barcode | TCCTTTCCTGATAGG | 41 |
| | | 3’ | NNNNNNNNNGCTTTAAGGCCGGTCCTAGC*A*A | 42 |
| B7 | Hu.CD14 | 5’ | GTGACTGGAGTTCAGACGTGTGCTCTTCCGATCTNNNN | 43 |
| | | | NNNNNN | |
| | | barcode | TCTCAGACCTCCGTA | 44 |
| | | 3’ | NNNNNNNNNGCTTTAAGGCCGGTCCTAGC*A*A | 45 |
| B8 | Hu.CD8 | 5’ | GTGACTGGAGTTCAGACGTGTGCTCTTCCGATCTNNNN | 46 |
| | | | NNNNNN | |
| | | barcode | GCGCAACTTGATGAT | 47 |
| | | 3’ | NNNNNNNNNGCTTTAAGGCCGGTCCTAGC*A*A | 48 |
| B9 | Hu.CD45 | 5’ | GTGACTGGAGTTCAGACGTGTGCTCTTCCGATCTNNNN | 49 |
| | | | NNNNNN | |
| | | barcode | TCCCTTGCGATTTAC | 50 |
| | | 3’ | NNNNNNNNNGCTTTAAGGCCGGTCCTAGC*A*A | 51 |
| B10 | Hu.ZAP70 | 5’ | GTGACTGGAGTTCAGACGTGTGCTCTTCCGATCTNNNN | 52 |
| | | | NNNNNN | |
| | | barcode | CTTGCCTGCAATCAC | 53 |
| | | 3’ | NNNNNNNNNGCTTTAAGGCCGGTCCTAGC*A*A | 54 |
| B11 | Hu.Perforin | 5’ | GTGACTGGAGTTCAGACGTGTGCTCTTCCGATCTNNNN | 55 |
| | | | NNNNNN | |
| | | barcode | TCTTCGTTTGAACGG | 56 |
| | | 3’ | NNNNNNNNNGCTTTAAGGCCGGTCCTAGC*A*A | 57 |
| B12 | Hu.GrzmB | 5’ | GTGACTGGAGTTCAGACGTGTGCTCTTCCGATCTNNNN | 58 |
| | | | NNNNNN | |
| | | barcode | GCGTGTTGTGGTATT | 59 |
| | | 3’ | NNNNNNNNNGCTTTAAGGCCGGTCCTAGC*A*A | 60 |

A “phosphorothioate (PS) bond” is indicated with a * in the above sequences and refers to a phosphorothioate (PS) bond between the two nucleotide residues. A phosphorothioate bond substitutes a sulfur atom for a non-bridging oxygen atom in the phosphate backbone of an oligo.

### Example 2: Identification of Cells Based on Protein Expression

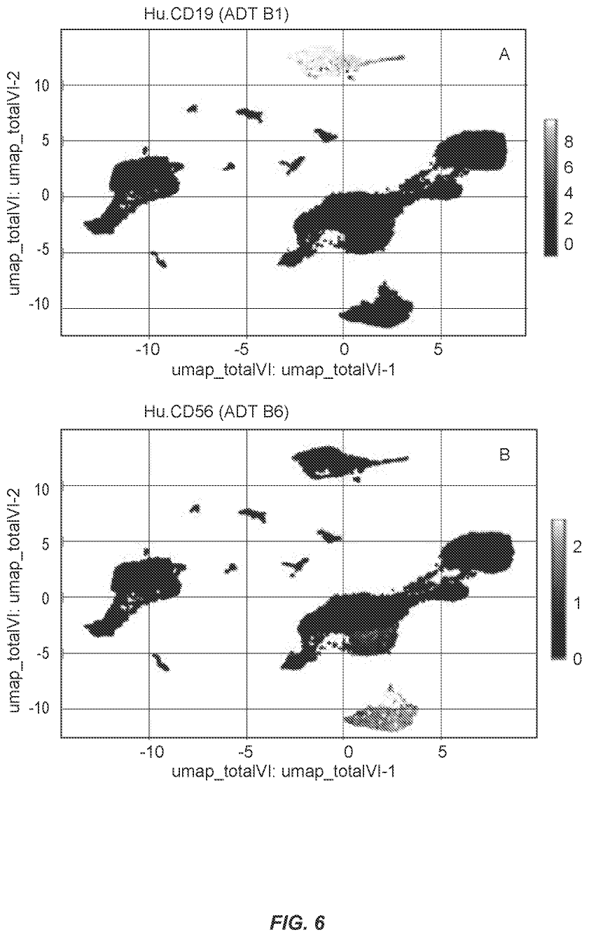

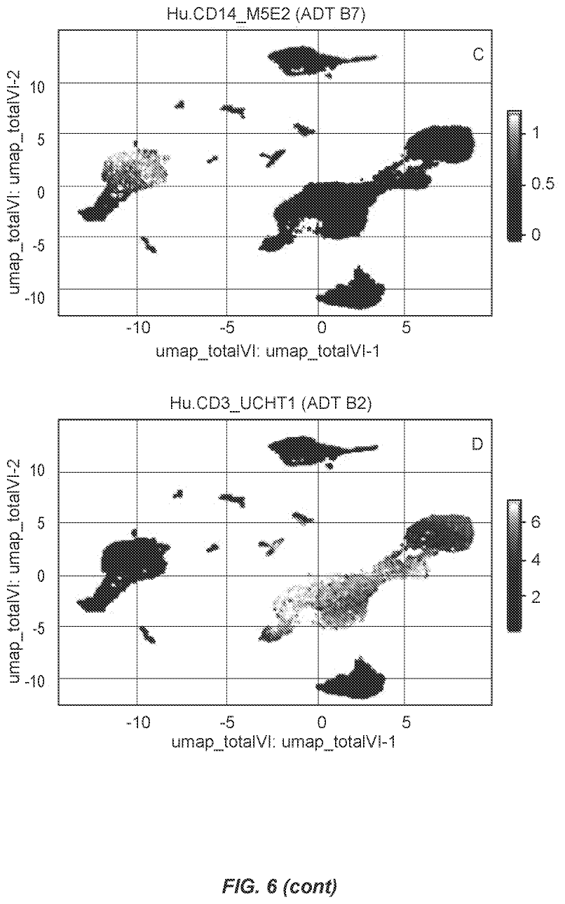

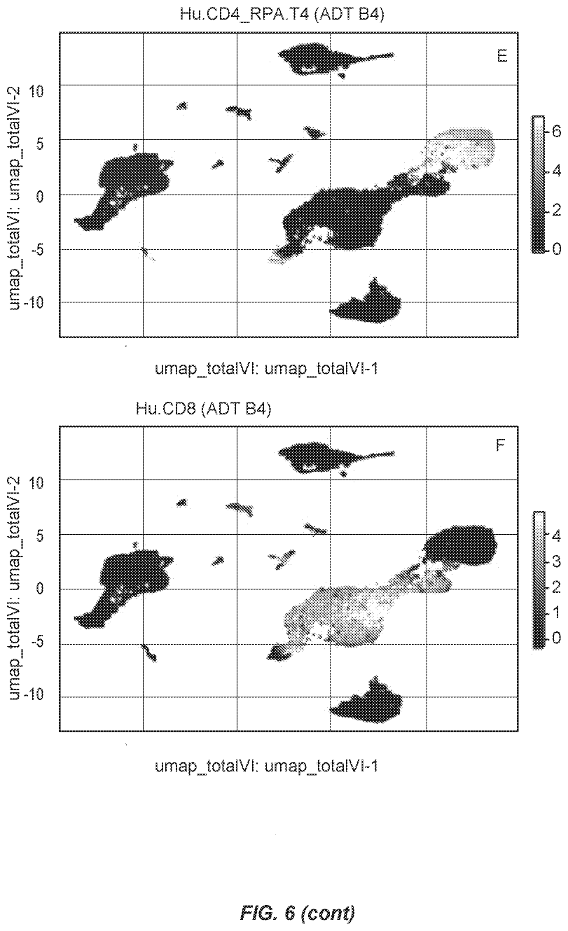

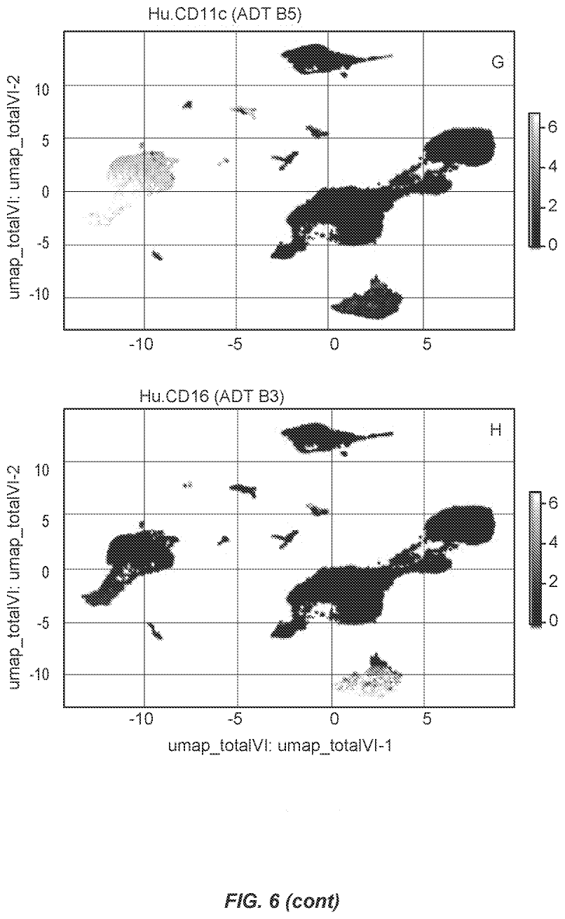

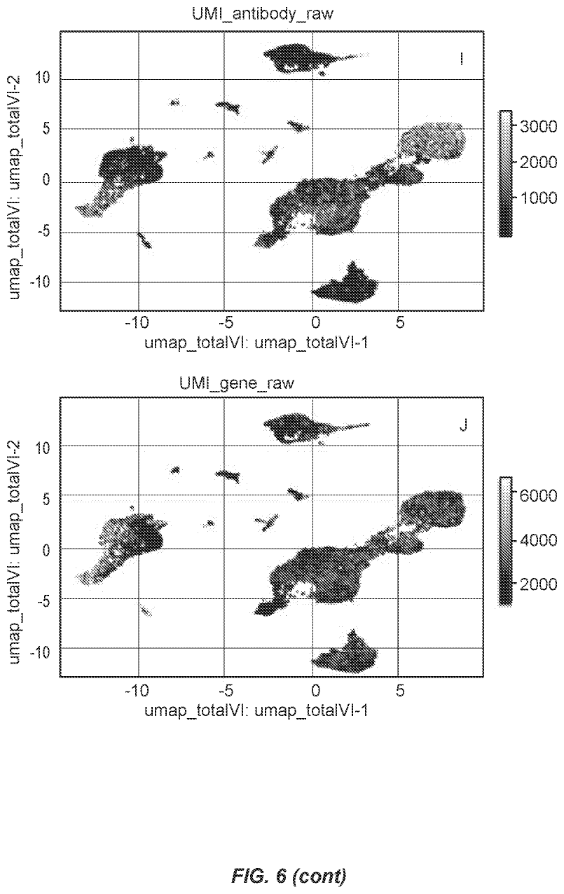

PBS is a common buffer used for cell analysis protocols, but this buffer is not capable of maintaining RNA integrity once the cells are fixed. Use of citrate buffer (e.g., saline-sodium citrate (SSC) buffer) in higher concentrations (3× and 5×) may be enough to bypass this issue allowing the detection of high quality single cell RNA (scRNA). However, how an increased osmolarity might affect intracellular protein detection remains unknown. In order to evaluate if a higher osmolarity is linked to positive results obtained with SSC 3× and negative results obtained with PBS 1×, different buffer compositions were tested to rehydrate methanol fixed/permeabilized cells. For this, PBMCs were isolated and stained with major surface markers that allow the characterization of T-cell, B-cell, natural killer (NK)-cell, and monocyte populations. Once stained and washed these cells were fixed with ice-cold methanol and rehydrated using either SSC 3× or PBS 3×. Additionally, BSA and SUPERASEIN concentrations were fixed (both at 1%) while testing different concentrations of DTT (1 mM, 10 mM and 40 mM) in order to identify an ideal concentration that prevents RNA degradation, and allows SUPERASEIN to function properly while maintaining antibody structure. As observed in FIG. 6, all buffers produced quality RNA without compromising surface antibody detection. All detected clusters were within the expected biological ranges and yielded similar results to the clusters obtained from unfixed cells. By using a differential expression based principal component analysis (PCA), SSC 3× with DTT 1 mM was identified as the ideal buffer due to the high similarity when compared against fresh cells.

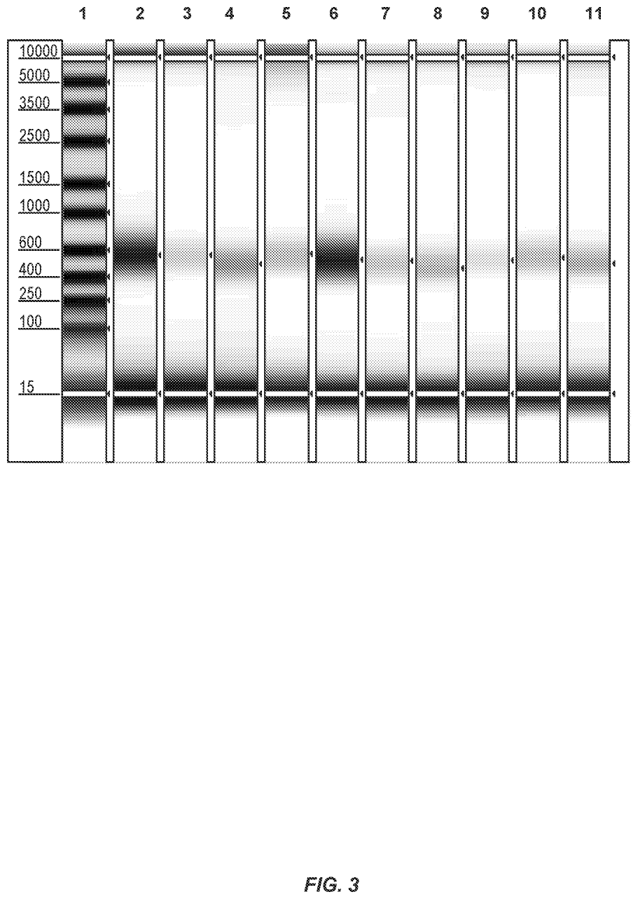

Since the buffer identified above worked well for methanol fixed cells, the same buffer was tested to determine whether it would work for paraformaldehyde (PFA) fixation. PFA fixation is harsher than methanol and no protocol exists showing how to obtain RNA after using this fixative. Once again, PBMCs were isolated and surface stained with TBNK markers (BIOLEGEND) and fixed with methanol or PFA (4% or 1%) for 1 hour on ice. As seen in FIG. 3, cDNA was detected from all three tested fixation condition but PFA concentration seems to be a key factor and 1% PFA yielded more cDNA than 4% PFA. Thus, 1% PFA was used for further experiments with permeabilization.

Unlike methanol, PFA requires permeabilization. Different detergents were added to the rehydration buffer (SSC 3×, BSA 1%, SUPERASEIN 1%, DTT 1 mM) to test each permeabilizing agent. TBNK surface stained PBMCs were fixed with methanol (used this time as a positive control for fixation and permeabilization) at −20° C. or PFA 1% for 1 hour on ice. The PFA fixed samples were washed with wash buffer (SSC 3×, BSA 1%, DTT 1 mM) to remove fixative, and cell pellets were resuspended in each permeabilization buffer tested (methanol 100%, Saponin 0.5%, TWEEN 20 0.2%, TRITON X-100 0.1% or NP-40 0.1%). After permeabilization, cells were blocked with MONOCYTE BLOCKER (BIOLEGEND) and FC block followed by intracellular staining for Perforin, Zap70 and GranzymeB antibodies, and submitted for a single cell sequencing protocol. The following protocols and buffers were used for these experiments.

### Permeabilization Protocol

1. Dilute stained cells to 5e6 cell/mL in PBS and aliquot 200 μL per tube (6 tubes total)

2. Fix the first tube (tube 1) with 800 μL pre-chilled MeOH leave it on −20° C. for >30 min

3. Spin remaining tubes for 1750 rpm 5 min 4° C., remove PBS and fix cells with 100 μL of PFA1% solution on ice for 1 h

4. Wash cells with 3 mL of wash buffer I at 1000×g 5 min 4° C.

5. Resuspend the cells from tube 2 in 500 μL of cold MeOH and leave it on −20° C. for >30 min

6. Resuspend the cells from tube 3 with 100 μL saponin solution for 30 min

7. Resuspend the cells from tube 4 with 100 μL 0.2% TWEEN-20 solution for 30 min

8. Resuspend the cells from tube 5 with 100 μL 0.1% TRITON X-100 solution for 15 min

9. Resuspend the cells from tube 6 with 100 μL 0.1% NP-40 solution for 15 min

10. Wash all tubes with 3 mL of wash buffer I and wash buffer II (tube 3 only)

11. Resupend cells in 50 uL of blocking diluted in wash buffer I or II and incubate for 15 min on ice

12. Add antibody cocktail diluted in wash buffer I or II and incubate for 30 min on ice

13. Wash cells 3 times with wash buffer I or II

14. Count cells and load 10× Chromium

Buffers

| Diluted Saponin (10%) | Diluted TWEEN-20 (20%) | Diluted TRITON X-100 (10%) | Diluted NP-40 (10%) |
|---|---|---|---|
| Wash Buffer I | Wash Buffer II | Rehydration buffer | PFA 1% solution |
| SSC 3X | Wash Buffer I | Wash Buffer I | Rehydration buffer |
| BSA 1% | Saponin 0.5% | SUPERASEIN 1% | PFA 1% |
| DTT 1 mM | Volume needed: 15 mL | Volume needed: 1 mL | Volume needed: 550 μL |
| Volume needed: 100 mL | | | |
| Saponin solution | TWEEN-20 solution | TRITON X-100 solution | NP-40 solution |
| Wash Buffer II | Rehydration buffer | Rehydration buffer | Rehydration buffer |
| 1% SUPERASEIN | TWEEN-20 0.2% | TRITON X-100 0.1% | NP-40 (Igepal) 0.1% |
| Volume needed: 125 μL | Volume needed: 125 μL | Volume needed: 125 μL | Volume needed: 125 μL |

Reverse Transcription (RT) Protocol (for Generating cDNA)

1. Cells were counted after staining and washed using CountesII device

2. A master mix containing RT buffer, reducing agent, reverse transcriptase enzyme, and template switch oligo or Poly-dT RT primer was prepared and kept on ice until use

3. Cells and master mix were combined following 10× protocol recommendation and loaded onto the chip

4. Beads were added

5. Partitioning oil was added (this creates an emulsion with the cells+master mix which is in aqueous solution) and the chip was loaded in the 10× chromium device

6. Once the GEMs (droplets in the emulsion) were done, the sample was transferred to PCR tubes and loaded in a thermocycler for the reverse transcription reaction to take place

| | |
|---|---|
| 53° C. | 45 min |
| 85° C. | 5 min |
| 4° C. | stored at this temperature until processed |

Variations to this protocol may include one or more of the following:

A. Adding a 65° C. incubation before the 53° C. step

B. Doing the whole process at 65° C.

C. Increasing the reducing agent concentration (e.g., for DSP fixation)

D. Increasing the concentration of reverse transcriptase enzyme

E. Increasing the incubation period (e.g., 10 min 65° C.+45 min 53° C. vs. 1 h 65° C.+45 min 53° C.).

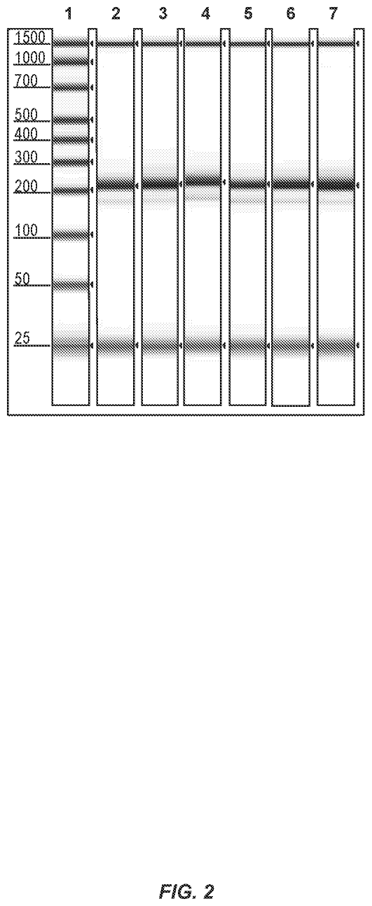

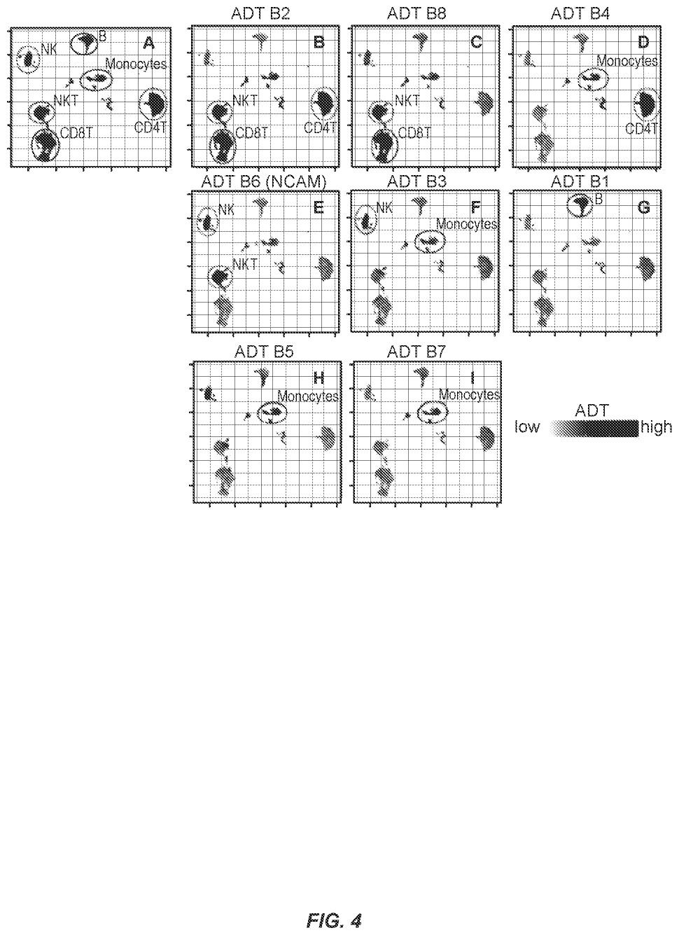

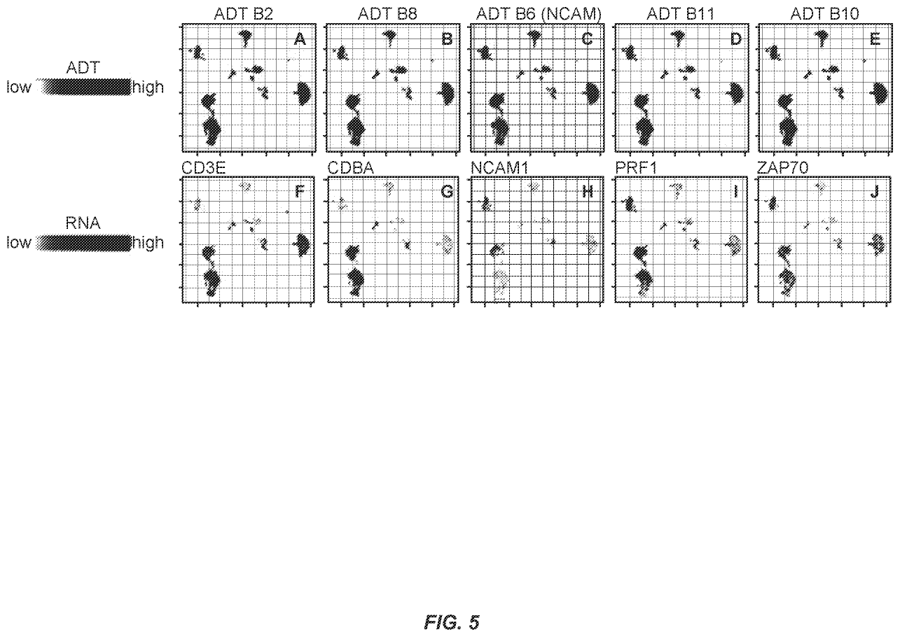

According to FIG. 2, there were no major differences during QC step for antibody-derived tags (ADTs) and all tested buffers yielded similar amplification efficiency. Nonetheless, the same was not observed for the cDNA amplification QC. As observed in FIG. 3, methanol fixed cells (lane 6) had the highest cDNA yield, and the combination of PFA fixation with methanol permeabilization gave the best result for the intracellular antibodies tested. After sequencing, the data was processed and all the expected cell clusters were identified in PBMCs (FIG. 4). As shown in FIG. 5, Perforin and Zap70 had a very strong correlation between protein (ADT) and cDNA pattern staining specifically NK, NKT and CD8 T cells (Perforin) and NK, NKT, CD4 and CD8 T cells (Zap70).

In view of the results above, certain conclusions include:
SSC 3× works better than PBS 3×
DTT 1 mM is the ideal condition to allow intracellular staining while maintaining intact surface antibodies
PFA 1% fixation followed by methanol permeabilization was the best condition for Perforin, Zap70 and Granzyme B antibodies
Saponin, TRITON X-100 and NP-40 produced acceptable results and may be useful for other antibodies
TWEEN 20 produced the worst results. Less cDNA was detected compared to the others, and background staining was higher than the rest.

### Example 3: Examples of Embodiments

A1. A method for detecting one or more targets in a sample, the method comprising contacting the sample with one or more of:

(i) a composition comprising a first construct that comprises a first ligand attached or conjugated to a polymer construct by a linker, said first ligand binding specifically to a first target, and said polymer construct comprising: an amplification handle; a barcode that specifically identifies said first ligand; an optional unique molecular identifier that is positioned adjacent to the barcode on its 5′ or 3′ end; and an anchor for hybridizing to a capture sequence that comprises a sequence complementary to said anchor;

(ii) a composition comprising at least one additional construct, which construct comprises an additional ligand attached or conjugated to an additional polymer construct by a linker, said additional ligand binding specifically to an additional target, and said additional polymer construct comprising an amplification handle; an additional barcode that specifically identifies said additional ligand; an optional additional unique molecular identifier that is positioned adjacent to the additional barcode on its 5′ or 3′ end, and an anchor for hybridizing to a capture sequence that comprises a sequence complementary to said anchor; and/or

(iii) a composition comprising one or more substantially identical constructs, each substantially identical construct differing from any other reference first or additional construct in the sequence of its optional unique molecular identifier (UMI) or the absence of the UMI.

A1.1 The method of embodiment A1, wherein the sample is a biological sample.

A2. The method of embodiment of A1 or A1.1, further comprising washing the sample to remove unbound constructs of the contacting step.

A3. The method of embodiment A1, A1.1 or A2, further comprising:

hybridizing the anchor sequence to a capture oligonucleotide sequence comprising a sequence complementary to said anchor and generating double stranded oligonucleotide sequences;

extending the capture oligonucleotide hybridized to the anchor sequence to copy the construct barcode, UMI and amplification handle onto the double stranded sequences; and

amplifying or detecting the sequences.

A4. The method of embodiment A3, wherein said amplifying or detecting comprises detecting the construct barcode sequences to identify whether the sample expresses or contains the first target, the additional target, or a combination of first target and additional target.

A5. The method of embodiment A3, wherein said amplifying or detecting comprises determining the expression level of the first target or additional target in the sample by detecting the amount of the corresponding construct barcodes normalized by the amount of any one of unique molecular identifiers or the mean amount of two or more of unique molecular identifiers.

A6. The method of any of embodiments A1 to A5, further comprising inserting one or more unique molecular identifiers adjacent each construct's barcode on its 5′ or 3′ end.

A7. The method of embodiment A6, wherein the contacting step comprises adding one or more of the compositions of claims 1 to 24 to said sample simultaneously or sequentially.

A8. The method of any of embodiments A3 to A7, further comprising isolating individual cells or populations of cells from the sample that are bound to one or more said first or additional constructs before the hybridizing step.

A9. The method of any of embodiments A3 to A8, wherein the extending step further comprises amplifying the double strand oligonucleotide sequences with primers annealed to the amplification handles.

A10. The method of any of embodiments A1 to A9, wherein the method is a high throughput method.

A11. The method of any one of embodiments A1 to A10, wherein the capture sequence is immobilized on a substrate.

A12. The method of embodiment A11, wherein the substrate is a bead, a slide, a multi-well plate, a microwell, a nanowell, or a chip.

A13. The method of embodiment A11 or A12, wherein the capture sequence further comprises an additional amplification handle; an additional barcode that specifically identifies the substrate to which the capture sequence is bound; and an optional additional unique molecular identifier that is positioned adjacent the additional barcode on its 5′ or 3′ end that identifies each capture sequence.

A14. The method of any one of embodiments A1 to A13, wherein said sample is a population of the same or a mixture of different cells, cell or cell membrane components, tissue, or a lysate of said cells or tissue.

B1. A high throughput method for detecting one or more targets in a sample, the method comprising contacting the sample with one or more of

(i) a composition comprising a first construct that comprises a first ligand that binds specifically to a first target, said first ligand attached or conjugated to a first polymer construct by a linker, wherein the first polymer construct comprises: an amplification handle; a barcode sequence that specifically identifies said first ligand from any other ligand that recognizes a different target, an optional unique molecular identifier sequence that is positioned adjacent to the 5′ or 3′ end of the barcode, and an anchor sequence for hybridizing to a capture sequence that comprises a sequence complementary to said anchor;

(ii) a composition of (i) comprising at least one additional construct, which comprises an additional ligand attached or conjugated to an additional polymer construct by a linker, said additional ligand binding specifically to an additional target, and said additional polymer construct comprising: an amplification handle; an additional barcode that specifically identifies said additional ligand; an optional additional unique molecular identifier that is positioned adjacent to the 5′ or 3′ end of the additional barcode, and an anchor sequence of (i), wherein said additional construct differs from any other construct in the composition in its antibody, target, barcode, and UMI; and/or

(iii) a composition of (i) or (ii) comprising one or more substantially identical constructs, each substantially identical construct differing from any other reference first or additional construct in the sequence of its optional unique molecular identifier (UMI) or the absence of the UMI.

B1.1 A high throughput method for detecting one or more targets in a sample, the method comprising contacting the sample under hybridization conditions with one or more of

(i) a composition comprising a first construct that comprises a first ligand that binds specifically to a first target, said first ligand attached or conjugated to a first polymer construct by a linker, wherein the first polymer construct comprises: an amplification handle; a barcode sequence that specifically identifies said first ligand from any other ligand that recognizes a different target, an optional unique molecular identifier sequence that is positioned adjacent to the 5′ or 3′ end of the barcode, and an anchor sequence for hybridizing to a capture sequence that comprises a sequence complementary to said anchor;

(ii) a composition of (i) comprising at least one additional construct, which comprises an additional ligand attached or conjugated to an additional polymer construct by a linker, said additional ligand binding specifically to an additional target, and said additional polymer construct comprising: an amplification handle; an additional barcode that specifically identifies said additional ligand; an optional additional unique molecular identifier that is positioned adjacent to the 5′ or 3′ end of the additional barcode, and an anchor sequence of (i), wherein said additional construct differs from any other construct in the composition in its antibody, target, barcode, and UMI; and/or

(iii) a composition of (i) or (ii) comprising one or more substantially identical constructs, each substantially identical construct differing from any other reference first or additional construct in the sequence of its optional unique molecular identifier (UMI) or the absence of the UMI; wherein the hybridization conditions comprise a hybridization buffer comprising a buffering agent, a stabilizing agent, a reducing agent, and blocking agent.

B1.2 The method of embodiment B1 or B1.1, wherein the sample is a biological sample.

B1.3 The method of embodiment B1, B1.1 or B1.2, wherein the ligand is an antibody or fragment thereof.

B1.3. The method of embodiment B1.3, wherein the target is an epitope.

B2. The method of any one of embodiments B1 to B1.3, further comprising washing the sample to remove unbound constructs.

B3. The method of any one of embodiments B1 to B2, further comprising:

annealing said construct anchor sequences to the capture oligonucleotide sequences of the contacted sample and generating a double stranded oligonucleotide sequence.

B4. The method of any one of embodiments B1 to B3 further comprising:

extending the capture oligonucleotide hybridized to the anchor sequence to copy the construct barcode, UMI and amplification handle onto the double stranded sequences; and amplifying or detecting the sequences.

B5. The method of embodiment B4, wherein said amplifying step comprises detecting the construct barcode sequences to identify whether the sample expresses or contains the first target, the additional target, or a combination of first target and additional target.

B6. The method of embodiment B4, wherein said amplifying step comprises determining the expression level of the first target or additional target in the sample by detecting the amount of the corresponding construct barcodes is normalized by the amount of any one of the unique molecular identifiers or the mean amount of two or more of unique molecular identifiers.

B7. The method of any one of embodiments B1 to B6, further comprising inserting one or more unique molecular identifiers adjacent each construct's barcode on its 5′ or 3′ end.

B8. The method of embodiment B1, wherein the contacting step further comprises adding the compositions (i), (ii), (iii) to said sample simultaneously or sequentially.

B9. The method of any one of embodiments B1 to B8, further comprising isolating individual cells, cell or membrane components, tissues or populations of same from the sample that are bound to one or more said first or additional constructs further analysis.

B10. The method of embodiment B4, wherein the extending step further comprises amplifying the double strand oligonucleotide sequences with primers annealed to the amplification handles.

B11. The method of any one of embodiments B1 to B10, wherein the capture sequence is immobilized on a substrate.

B12. The method of embodiment B11, wherein the substrate is a bead, a slide, a multi-well plate, a microwell, a nanowell, or a chip.

B13. The method of any one of embodiments B1 to B12, wherein the capture sequence further comprises an amplification handle; a barcode that specifically identifies a specific substrate; and an optional additional unique molecular identifier that is positioned adjacent to the amplification handle on its 3′end or the said barcode on its 3′ end.

B14. The method of any one of embodiments B1.1 to B13, wherein the buffering agent is saline-sodium citrate or phosphate buffer saline.

B15. The method of any one of embodiments B1.1 to B14, wherein the buffering agent is saline-sodium citrate, 4-(2-hydroxyethyl)-1-piperazineethanesulfonic acid (HEPES), SSPE, piperazine-N,N′-bis(2-ethanesulfonic acid) (PIPES), tetramethyl ammonium chloride (TMAC), Tris(hydroxymethyl)aminomethane (Tris), SET, citric acid, potassium phosphate, sodium pyrophosphate, and combinations thereof.

B16. The method of any one of embodiments B1.1 to B15, wherein the stabilizing agent is a serum solution.

B17. The method of embodiment B16, wherein the serum solution comprises fetal bovine serum, human serum, or bovine serum albumin.

B18. The method of any one of embodiments B1.1 to B17, wherein the reducing agent is dithiothreitol.

B19. The method of any of embodiments B1.1 to B18, wherein the blocking agent is an oligonucleotide.

B20. The method of any one of embodiments B1.1 to B19, wherein the blocking agent is a phosphorothioate oligonucleotide.

B21. The method of any of embodiments B1.1 to B18, wherein the blocking agent comprises single-stranded binding proteins.

C1. A high throughput method for characterizing a cell by simultaneous detection of one or more targets located in or on the cell and the transcriptome, genome, or epigenome, the method comprising contacting a sample containing cells with one or more of:

(i) a composition that comprises a first construct that comprises a first ligand that binds specifically to a first target located in or on the surface of a cell, said first ligand conjugated to a first polymer construct by a linker, wherein the first polymer construct comprises an amplification handle; a barcode sequence that specifically identifies said first ligand from any other ligand that recognizes a different target, an optional unique molecular identifier sequence that is positioned adjacent the 5′ or 3′ end of the barcode, and a polyA anchor sequence designed for hybridizing to a capture oligonucleotide sequence comprising a polyT sequence immobilized on a microfluidics bead, a slide, a microwell, or a nanowell;

(ii) a composition of (i) comprising at least one additional construct, which comprises an additional ligand conjugated to an additional polymer construct by a linker, said additional ligand binding specifically to an additional target, and said additional polymer construct comprising: the amplification handle of (i); an additional barcode that specifically identifies said additional ligand; an optional additional unique molecular identifier that is positioned adjacent the 5′ or 3′ end of the additional barcode, and the said anchor of (i), wherein said additional ligand, additional barcode, additional UMI and additional target differ from the corresponding components in any other construct in the composition; and/or

(iii) a composition of (i) or (ii) comprising one or more substantially identical constructs, each substantially identical construct differing from any other reference first or additional construct in the sequence of its optional unique molecular identifier (UMI) or the absence of the UMI.

C1.1 A high throughput method for characterizing a cell by simultaneous detection of one or more targets located in or on the cell and the transcriptome, genome, or epigenome, the method comprising contacting a sample containing cells under hybridization conditions with one or more of:

(i) a composition that comprises a first construct that comprises a first ligand that binds specifically to a first target located in or on the surface of a cell, said first ligand conjugated to a first polymer construct by a linker, wherein the first polymer construct comprises an amplification handle; a barcode sequence that specifically identifies said first ligand from any other ligand that recognizes a different target, an optional unique molecular identifier sequence that is positioned adjacent the 5′ or 3′ end of the barcode, and a polyA anchor sequence designed for hybridizing to a capture oligonucleotide sequence comprising a polyT sequence immobilized on a microfluidics bead, a slide, a microwell, or a nanowell;

(ii) a composition of (i) comprising at least one additional construct, which comprises an additional ligand conjugated to an additional polymer construct by a linker, said additional ligand binding specifically to an additional target, and said additional polymer construct comprising: the amplification handle of (i); an additional barcode that specifically identifies said additional ligand; an optional additional unique molecular identifier that is positioned adjacent the 5′ or 3′ end of the additional barcode, and the said anchor of (i), wherein said additional ligand, additional barcode, additional UMI and additional target differ from the corresponding components in any other construct in the composition; and/or

(iii) a composition of (i) or (ii) comprising one or more substantially identical constructs, each substantially identical construct differing from any other reference first or additional construct in the sequence of its optional unique molecular identifier (UMI) or the absence of the UMI; wherein the hybridization conditions comprise a hybridization buffer comprising a buffering agent, a stabilizing agent, a reducing agent, and blocking agent.

C1.2 The method of embodiment C1 or C1.1, wherein the sample is a biological sample.

C1.3 The method of embodiment C1, C1.1 or C1.2, wherein the ligand is an antibody or fragment thereof.

C1.3. The method of embodiment C1.3, wherein the target is an epitope.

C2. The method of any one of embodiments C1 to C1.3, further comprising:

encapsulating an individual single cell bound to one or more constructs into an aqueous droplet with one said bead, wherein each bead is conjugated to the capture sequence comprising a unique bead barcode sequence, an optional UMI, and a 3′ polyT sequence.

C3. The method of embodiment C2, further comprising:

lysing the single cell in each droplet, wherein mRNAs in the cell and said polymer construct released from the ligand anneal to the polyT sequences of the capture sequence; and

generating from the sequences annealed to the bead (A) double stranded cDNAs containing the bead barcode sequence and the reverse transcripts of the cellular mRNA and (B) a double stranded DNA containing the bead barcode sequence and the polymer construct.

C4. The method of embodiment C3, further comprising creating by amplification a library comprising the cDNA of (A) and the DNA containing the polymer construct of (B); and detecting the sequences.

C5. The method of embodiment C4, wherein the detecting step comprises detecting the construct barcode sequences to identify whether the single cell expresses the first target.

C6. The method of embodiment C4, wherein the detecting step comprises determining the expression level of the first or additional target in the single cell by detecting the amount of the construct barcode normalized by the amount of any of the unique molecular identifiers or the mean amount of two or more unique molecular identifiers.

C7. The method of embodiment C5 or C6, further comprising associating the transcriptome, genome, or epigenome; or components of the transcriptome, genome, or epigenome of the library with the cell on which the target was identified.

C8. The method of any one of embodiments C1 to C7, wherein the contacting step further comprises adding the compositions (i), (ii), (iii) to said sample simultaneously or sequentially.

C9. The method of any one of embodiments C1.1 to C8, wherein the buffering agent is saline-sodium citrate or phosphate buffer saline.

C10. The method of any one of embodiments C1.1 to C9, wherein the buffering agent is saline-sodium citrate, 4-(2-hydroxyethyl)-1-piperazineethanesulfonic acid (HEPES), SSPE, piperazine-N,N′-bis(2-ethanesulfonic acid) (PIPES), tetramethyl ammonium chloride (TMAC), Tris(hydroxymethyl)aminomethane (Tris), SET, citric acid, potassium phosphate, sodium pyrophosphate, and combinations thereof.

C11. The method of any one of embodiments C1.1 to C10, wherein the stabilizing agent is a serum solution.

C12. The method of embodiment C11, wherein the serum solution comprises fetal bovine serum, human serum, or bovine serum albumin.

C13. The method of any one of embodiments C1.1 to C12, wherein the reducing agent is dithiothreitol.

C14. The method of any of embodiments C1.1 to C13, wherein the blocking agent is an oligonucleotide.

C15. The method of any one of embodiments C1.1 to C14, wherein the blocking agent is a phosphorothioate oligonucleotide.

C16. The method of any of embodiments C1.1 to C13, wherein the blocking agent comprises single-stranded binding proteins.

D1. A hybridization buffer composition comprising a buffering agent, a stabilizing agent, a reducing agent, and blocking agent.

D2. The composition of embodiment D1, wherein the buffering agent is saline-sodium citrate or phosphate buffer saline.

D3. The composition of embodiment D1 or D2, wherein the buffering agent is saline-sodium citrate, 4-(2-hydroxyethyl)-1-piperazineethanesulfonic acid (HEPES), SSPE, piperazine-N,N′-bis(2-ethanesulfonic acid) (PIPES), tetramethyl ammonium chloride (TMAC), Tris(hydroxymethyl)aminomethane (Tris), SET, citric acid, potassium phosphate, sodium pyrophosphate, and combinations thereof.

D4. The composition of any one of embodiments D1 to D3, wherein the stabilizing agent is a serum solution.

D5. The composition of embodiment D4, wherein the serum solution comprises fetal bovine serum, human serum, or bovine serum albumin.

D6. The composition of any one of embodiments D1 to D5, wherein the reducing agent is dithiothreitol.

D7. The composition of any of embodiments D1 to D6, wherein the blocking agent is an oligonucleotide.

D8. The composition of any one of embodiments D1 to D7, wherein the blocking agent is a phosphorothioate oligonucleotide.

E1. A method for detecting one or more targets in a sample, the method comprising contacting the sample under hybridization conditions with one or more of:

(i) a composition comprising a first construct that comprises a first ligand attached or conjugated to a polymer construct by a linker, said first ligand binding specifically to a first target, and said polymer construct comprising: an amplification handle; a barcode that specifically identifies said first ligand; an optional unique molecular identifier that is positioned adjacent to the barcode on its 5′ or 3′ end; and an anchor for hybridizing to a capture sequence that comprises a sequence complementary to said anchor;

(ii) a composition comprising at least one additional construct, which construct comprises an additional ligand attached or conjugated to an additional polymer construct by a linker, said additional ligand binding specifically to an additional target, and said additional polymer construct comprising an amplification handle; an additional barcode that specifically identifies said additional ligand; an optional additional unique molecular identifier that is positioned adjacent to the additional barcode on its 5′ or 3′ end, and an anchor for hybridizing to a capture sequence that comprises a sequence complementary to said anchor; and/or

(iii) a composition comprising one or more substantially identical constructs, each substantially identical construct differing from any other reference first or additional construct in the sequence of its optional unique molecular identifier (UMI) or the absence of the UMI;

wherein the hybridization conditions comprise a hybridization buffer comprising a buffering agent, a stabilizing agent, a reducing agent, and blocking agent.

E1.1 The method of embodiment E1, wherein the sample is a biological sample.

E2. The method of embodiment of E1 or E1.1, further comprising washing the sample to remove unbound constructs of the contacting step.

E3. The method of embodiment E1, E1.1 or E2, further comprising:

hybridizing the anchor sequence to a capture oligonucleotide sequence comprising a sequence complementary to said anchor and generating double stranded oligonucleotide sequences;

extending the capture oligonucleotide hybridized to the anchor sequence to copy the construct barcode, UMI and amplification handle onto the double stranded sequences; and

amplifying or detecting the sequences.

E4. The method of embodiment E3, wherein said amplifying or detecting comprises detecting the construct barcode sequences to identify whether the sample expresses or contains the first target, the additional target, or a combination of first target and additional target.

E5. The method of embodiment E3, wherein said amplifying or detecting comprises determining the expression level of the first target or additional target in the sample by detecting the amount of the corresponding construct barcodes normalized by the amount of any one of unique molecular identifiers or the mean amount of two or more of unique molecular identifiers.

E6. The method of any of embodiments E1 to E5, further comprising inserting one or more unique molecular identifiers adjacent each construct's barcode on its 5′ or 3′ end.

E7. The method of embodiment E6, wherein the contacting step comprises adding one or more of the compositions of claims 1 to 24 to said sample simultaneously or sequentially.

E8. The method of any of embodiments E3 to E7, further comprising isolating individual cells or populations of cells from the sample that are bound to one or more said first or additional constructs before the hybridizing step.

E9. The method of any of embodiments E3 to E8, wherein the extending step further comprises amplifying the double strand oligonucleotide sequences with primers annealed to the amplification handles.

E10. The method of any of embodiments E1 to E9, wherein the method is a high throughput method.

E11. The method of any one of embodiments E1 to E10, wherein the capture sequence is immobilized on a substrate.

E12. The method of embodiment E11, wherein the substrate is a bead, a slide, a multi-well plate, a microwell, a nanowell, or a chip.

E13. The method of embodiment E11 or E12, wherein the capture sequence further comprises an additional amplification handle; an additional barcode that specifically identifies the substrate to which the capture sequence is bound; and an optional additional unique molecular identifier that is positioned adjacent the additional barcode on its 5′ or 3′ end that identifies each capture sequence.

E14. The method of any one of embodiments E1 to E13, wherein said sample is a population of the same or a mixture of different cells, cell or cell membrane components, tissue, or a lysate of said cells or tissue.

E15. The method of any one of embodiments E1 to E14, wherein the buffering agent is saline-sodium citrate or phosphate buffer saline.

E16. The method of any one of embodiments E1 to E15, wherein the buffering agent is saline-sodium citrate, 4-(2-hydroxyethyl)-1-piperazineethanesulfonic acid (HEPES), SSPE, piperazine-N,N′-bis(2-ethanesulfonic acid) (PIPES), tetramethyl ammonium chloride (TMAC), Tris(hydroxymethyl)aminomethane (Tris), SET, citric acid, potassium phosphate, sodium pyrophosphate, and combinations thereof.

E17. The method of any one of embodiments E1 to E16, wherein the stabilizing agent is a serum solution.

E18. The method of embodiment E17, wherein the serum solution comprises fetal bovine serum, human serum, or bovine serum albumin.

E19. The method of any one of embodiments E1 to E18, wherein the reducing agent is dithiothreitol.

E20. The method of any of embodiments E1 to E19, wherein the blocking agent is an oligonucleotide.

E21. The method of any one of embodiments E1 to E20, wherein the blocking agent is a phosphorothioate oligonucleotide.

E22. The method of any of embodiments E1 to E19, wherein the blocking agent comprises single-stranded binding proteins.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1.-40. (canceled)

41. A method of detecting one or more targets in a sample, comprising:
(a) contacting the sample with a permeabilization buffer;
(b) contacting a biological sample with an oligonucleotide blocking agent;
(c) contacting the biological sample with a composition comprising a first construct that comprises a first ligand attached or conjugated to a polymer construct by a linker, said first ligand binding specifically to a first target, wherein the polymer construct comprises a barcode sequence that specifically identifies the first ligand;
(d) contacting the biological sample with a composition comprising at least one additional construct, which construct comprises an additional ligand attached or conjugated to an additional polymer construct by a linker, said additional ligand binding specifically to an additional target, wherein the additional polymer construct comprises a barcode sequence that specifically identifies the additional ligand; and
(e) detecting the barcode sequences to identify whether the biological sample expresses or contains the first target, the additional target, or a combination of the first target and additional target.

42. The method of claim 41, wherein the one or more targets comprise intracellular targets.

43. The method of claim 41, wherein the one or more targets comprise cell surface targets.

44. The method of claim 41, wherein the targets comprise protein and/or nucleic acid targets.

45. The method of claim 41, wherein the polymer construct and additional polymer construct further comprise an amplification handle and an anchor for hybridizing to a capture sequence that comprises a sequence complementary to said anchor.

46. The method of claim 41, further comprising a unique molecular identifier that is positioned adjacent to the barcode on its 5′ or 3′ end.

47. The method of claim 41, further comprising hybridizing the anchor sequence to the capture oligonucleotide sequence comprising a sequence complementary to said anchor and generating double stranded oligonucleotide sequences prior to the detecting.

48. The method of claim 41, further comprising extending the capture oligonucleotide hybridized to the anchor sequence to copy the construct barcode and amplification handle onto the double stranded sequences prior to the detecting.

49. The method of claim 41, wherein the sample is a biological sample.

50. The method of claim 41, wherein the ligand is an antibody or fragment thereof.

51. The method of claim 41, wherein the oligonucleotide blocking agent comprises a phosphorothioate oligonucleotide.

52. The method of claim 41, wherein the oligonucleotide blocking agent comprises single-stranded binding proteins.

53. The method of claim 41, wherein the capture sequence is immobilized on a substrate.

54. The method of claim 53, wherein the substrate is a bead, a slide, a multi-well plate, a microwell, a nanowell, or a chip.

55. The method of claim 41, wherein said sample comprises a population of the same or a mixture of different cells, cell or cell membrane components, tissue, or a lysate of said cells or tissue.

56. The method of claim 41, further comprising fixation procedures before the contacting step or between sequential contacting steps with first or additional constructs.

57. The method of claim 41, further comprising a washing step.

58. The method of claim 41, wherein the detecting comprises determining the expression level of the first target or additional target in the sample by detecting the amount of the corresponding construct barcodes normalized by the amount of any one of unique molecular identifiers or the mean amount of two or more of unique molecular identifiers.

59. The method of claim 48, wherein the extending step further comprises amplifying the double strand oligonucleotide sequences with primers annealed to the amplification handles.

60. The method of claim 41, wherein said sample is a population of the same or a mixture of different cells, cell or cell membrane components, tissue, or a lysate of said cells or tissue.
